(19) United States
(12) Patent Application Publication
(10) Pub. No.: US 2025/0382228 A1
(43) Pub. Date: Dec. 18, 2025

(54) PRESS-FORMED BIOCEMENT PROCESSES, COMPOSITIONS, AND EQUIPMENT

(71) Applicant: Biomason Inc., Durham, NC (US)

(72) Inventors: Ginger K. Dosier; J. Michael Dosier, Raleigh, NC (US); Kimberly Anderson, Apex, NC (US); Chris Mekeel, Knightdale, NC (US)

(21) Appl. No.: 18/877,753

(22) PCT Filed: Jun. 22, 2023
(86) PCT No.: PCT/US23/68908

§ 371 Date: Dec. 20, 2024

Related U.S. Application Data
(60) Provisional application No. 63/354,543, filed on Jun. 22, 2022.
(60) Provisional application No. 63/380,642, filed on Oct. 24, 2022.

(51) Int. Cl. C04B 14/28; B01F 27/90; B01F 29/00; B01F 35/21; B01F 35/22; B01F 35/221; B01F 35/53; B01F 35/71; B01F 35/90; B01F 101/28; B28C 5/08; B28C 5/18; B28C 7/02; B28C 7/04; C04B 22/12; C04B 24/12; C04B 24/14; C04B 28/04; C04B 40/00; C04B 103/00; C04B 111/00; C12N 9/80

(52) U.S. Cl. CPC C04B 14/28; B01F 27/90; B01F 29/00; B01F 35/2115; B01F 35/2132; B01F 35/2135; B01F 35/2206; B01F 35/2215; B01F 35/5311; B01F 35/7176; B01F 35/90; B28C 5/0831; B28C 5/18; B28C 7/024; B28C 7/0409; B28C 7/0418; C04B 22/124; C04B 24/126; C04B 24/14; C04B 28/04; C04B 40/0067; C04B 40/0071; C04B 40/0082; C12N 9/80; C12Y 305/01005; B01F 2035/99; B01F 2101/28; B01F 2215/0431; B01F 2215/0454; C04B 2103/0001; C04B 2111/00017; C04B 2201/50

(57) ABSTRACT

Described herein are novel construction materials and construction material compositions, processes, and equipment for manufacturing press-formed biocement and bioconcrete products and construction materials. In some embodiments, the methods include combining aggregate particles with a first measured dose of at least one biological organism or enzyme and a first measured dose of cementation reagents in a first mixer, mixing the contents of the first mixer and reacting the first measured dose of the cementation reagents in the presence of the first measured dose of the at least one biological organism or enzyme to form a biocement which binds to the surfaces of the aggregate particles, thereby increasing the size of the particles to yield a biocement coated aggregate.

### CROSS-REFERENCE

This application is a 371 of international PCT/US2023/068908, filed Jun. 22, 2023, which claims the benefit of U.S. Provisional Application No. 63/354,543, filed Jun. 22, 2022 and U.S. Provisional Application No. 63/380,642, filed Oct. 24, 2022, each of which is incorporated herein by reference in its entirety.

### BACKGROUND

Biocement technologies offer cost effective high-strength building materials, structural materials, and concretes which have a substantially reduced carbon emission footprint compared to traditional building materials and concretes based on Portland cement. Biocement technologies often rely on hydroponics to produce strong bonds between particles of aggregate, which require complex processing steps and large solution volumes for recirculation of reagents and nutrients through cement or concrete molding forms. Accordingly, improved processes, compositions, and equipment are needed to promote large-scale adoption and production of biocement products, and replacement of conventional materials with reduced carbon-impact biocement materials.

### SUMMARY

In one aspect, described herein are methods of producing a bioconcrete construction materials. In some embodiments, the method comprises combining aggregate particles with a first measured dose of at least one biological organism or enzyme and a first measured dose of cementation reagents in a first mixer. In some embodiments, the method comprises mixing the contents of the first mixer and reacting the first measured dose of the cementation reagents in the presence of the first measured dose of the at least one biological organism or enzyme to form a biocement which binds to the surfaces of the aggregate particles, thereby increasing the size of the particles to yield a biocement coated aggregate.

In some embodiments, the method comprises combining a second measured dose of at least one biological organism or enzyme and a second measured dose of cementation reagents with the biocement coated aggregate in either the first mixer or a second mixer. In some embodiments, the method comprises compacting the mixture of the biocement coated aggregate, the second measured dose of at least one biological organism or enzyme, and the second measured dose of cementation reagents in a mold or form to reduce the volume of empty space between the biocement coated aggregate particles. In some embodiments, the method comprises reacting the second measured dose of cementation reagents in the presence of the second measured dose of at least one biological organism or enzyme to form biocement bridges between the biocement coated aggregate particles, thereby consolidating the biocement coated aggregate particles into a bioconcrete construction material.

In some embodiments, the first or second mixer is a thermally regulated to maintain a target temperature. In some embodiments, the total moisture content of the contents of the first or second mixer is less than about 25 wt %. In some embodiments, the total moisture content of the contents of the first or second mixer is controlled and to be less than about 15 wt %.

In some embodiments, the steps of the method are carried out in a temperature-controlled and humidity-controlled environment. In some embodiments, the temperature, humidity, and/or pH are monitored and controlled during either cementation reaction. In some embodiments, either of the first or second measured doses of at least one biological organism or enzyme comprises urease or cells of a urease-producing microorganism. In some embodiments, either of the first or second measured doses of at least one biological organism or enzyme comprises an acid-producing enzyme or cells of an acid-producing microorganism.

In some embodiments, either of the first or second measured doses of at least one biological organism or enzyme comprises carbonic anhydrase or cells of a carbonic anhydrase-producing microorganism.

In some embodiments, the urease-producing microorganism is Sporosarcina pasteurii. In some embodiments, the urease-producing microorganism is selected from the group of: Sporosarcina pasteurii, Sporosarcina ureae, Proteus vulgaris, Bacillus sphaericus, Myxococcus xanthus, Proteus mirabilis, Bacillus megaterium, Helicobacter pylori, and combinations of two or more thereof. In some embodiments, the cells comprise spores. In some embodiments, the biocement comprises bacterially precipitated calcium carbonate. In some embodiments, either of the first or second measured dose of cementation reagents comprises nutrients which promote the growth or enzymatic activities of microorganisms. In some embodiments, the nutrients comprise one or more of salts, amino acids, proteins, peptides, carbohydrates, saccharides, polysaccharides, fatty acids, oil, vitamins and minerals.

In some embodiments, either of the first or second measured dose of cementation reagents comprises a calcium source. In some embodiments, either of the first or second measured dose of cementation reagents comprises a urea source. In some embodiments, either of the first or second measured dose of cementation reagents comprises calcium carbonate. In some embodiments, either of the first or second measured dose of cementation reagents comprises calcium chloride. In some embodiments, either of the first or second measured dose of cementation reagents comprises cells of a urea-producing microorganism.

In some embodiments, the urea-producing microorganism is selected from the group of: Pseudomonas, Delaya avenusta, Thiosphacra pantotropha, Pseudomonas stutzen, Fragilaria crotonensis, Pseudoalteromonas spp., Pseudoalteromonas haloplanktis, Halomonas venusta, Pseudomonas balcarica, Pseudomonas stutzeri, Bacillus megaterium, Exiguobacterium aurantiacum, Pseudoalteromonas aliena, Pseudoalteromonas luteoviolacea, E. coli, and variants, serotypes, mutations, recombinant forms, and combinations thereof.

In some embodiments, the acid-producing microorganism is selected from the group consisting of: Variovorax, Klebsiella, Pseudomonas, Bacillus, Exiguobacterium, Microbacterium, Curtobacterium, Rathayibacter, CellFimi2, Streptomyces, Raoultella, B. pumilus, B. safanensis, B. simplex, B. licheniformis, and combinations thereof. In some embodiments, the acid produced by the acid-producing enzyme of the cells of the acid-producing microorganism is a carboxylic acid. In some embodiments, the acid produced by the acid-producing enzyme of the cells of the acid-producing microorganism is acetic acid, formic acid, propionic acid, butyric acid, gluconic acid, succinic acid, lactic acid, or citric acid. In some embodiments, compacting the mixture reduces the average linear distance between adjacent aggregate particles by at least about 25%.

In some embodiments, compacting the mixture produces an absolute packing efficiency of the aggregate particles of at least about 50%.

In some embodiments, compacting the mixture is performed by placing the mixture in a vibratory press and applying pressure and vibration to reduce the volume of empty space between the biocement coated aggregate particles. In some embodiments, the vibratory motor of the press is operated at a rotational speed from about 100 RPM to about 7200 RPM. In some embodiments, the vibratory motor of the press is operated at a duty cycle from 0.01% to about 100%. In some embodiments, the vibration and pressure are applied simultaneously. In some embodiments, the vibration and pressure are applied alternatively.

In some embodiments, the finished bioconcrete construction material comprises at least about 2% biocement by weight. In some embodiments, the finished bioconcrete construction material comprises at most about 20% biocement by weight.

In some embodiments, the aggregate particles comprise natural, non-natural, recycled or manufactured sand, ore, crushed rock, stone, minerals, crushed or fractured glass, wood, ash, foam, basalt, fibers, mine tailings, paper, waste materials, waste from a manufacturing process, plastics, polymers, roughened materials, and/or combinations thereof. In some embodiments, the bioconcrete construction material comprises bricks, thin bricks, pavers, panels, tile, veneer, cinder, breeze, clinker or aerated blocks, counter-tops, table-tops, design structures, blocks, a solid masonry structure, piers, foundations, beams, walls, or slabs.

In another aspect, described herein are mixing devices for biocement processing. In some embodiments, a mixing device described herein comprises a mixing tank. In some embodiments, a mixing device described herein comprises a plurality of mixing tines.

In some embodiments, a mixing device described herein comprises a mixing motor. In some embodiments the mixing motor is operably coupled to the mixing tines. In some embodiments, a mixing device described herein comprises a motor controller. In some embodiments, the motor controller is operably coupled to the mixing motor.

In some embodiments, a mixing device described herein comprises a reagent delivery pump. In some embodiments, the reagent delivery pump is operably coupled by a reagent line to infuse a quantity of a reagent from a reservoir into the mixing tank.

In some embodiments, a mixing device described herein comprises a pump controller, operably coupled to the reagent delivery pump. In some embodiments, a mixing device described herein comprises a heater operably coupled to the mixing tank. In some embodiments, a mixing device described herein comprises a temperature sensor configured to measure the temperature inside the mixing tank. In some embodiments, a mixing device described herein comprises a temperature controller, operably coupled to the temperature sensor and the heater. In some embodiments, a mixing device described herein comprises a main controller, operably coupled to the motor controller, pump controller, and temperature controller.

In some embodiments, the main controller is configured to facilitate production of biocement coated aggregate particles using the mixing device. In some embodiments, the motor controller is configurable to set the motor speed and the motor duty cycle. In some embodiments, the motor controller is configurable to set a total mixing time. In some embodiments, the temperature controller maintains a set temperature using a proportional-integral-derivative control algorithm based on a temperature value measured by the temperature sensor. In some embodiments, the reagent delivery pump further comprises a second temperature sensor, a second heater, and is operably coupled to the temperature controller.

In some embodiments, the mixing motor can be configured to operate at speeds from about 1 to about 120 RPM. In some embodiments, the duty cycle is configurable from about 0.01% to about 100%. In some embodiments, the duty cycle is configurable from about 0.05% to about 0.1%.

In some embodiments, the temperature controller is configurable to maintain a temperature of about 20° C. to about 40° C.

In some embodiments, the mixing tank has a mixing capacity of about 0.1 yd³ to about 30 yd³. In some embodiments, the mixing tank has a mixing capacity of at least about 4 yd³.

In some embodiments, the reagent delivery pump is configurable to deliver a fixed volume, a fixed mass, a fixed flow rate, or a custom flow pattern of reagent into the mixing tank.

In some embodiments, the mixing device further comprising a moisture sensor operably coupled to the main controller and configured to measure the moisture level inside the mixing tank. In some embodiments, the moisture level inside the tank is used to adjust the quantity of reagent delivered into the tank or is used to adjust the temperature controller.

In some embodiments, the heater is a heat-exchanger. In some embodiments, the heater is a resistive heater. In some embodiments, the heater is a forced-air heater. In some embodiments, the device maintains a homogeneous temperature inside the mixing tank, reagent reservoir, and reagent line at a set point of the temperature controller and is substantially free of hot spots.

In some embodiments, the reagent pump is a peristaltic pump, a syringe pump, a rotary vane pump, a venturi pump, or a diaphragm pump. In some embodiments, the mixing tines are attached to a mixing paddle or mixing wheel, which is mounted inside of the mixing tank and operably coupled to an output shaft of the mixing motor. In some embodiments, the mixing tines are attached to the walls of the mixing tank and the mixing tank comprises a rotatable drum which is operably coupled to an output shaft of the mixing motor.

In some embodiments, the reservoir is configured to deliver a reagent comprising a biological organism or enzyme, calcium, and urea. In some embodiments, the mixing tank further comprises a pH sensor or ion selective electrode for reaction monitoring, operably coupled to the main controller.

Another aspect of the present disclosure provides a system comprising one or more computer processors and computer memory coupled thereto. The computer memory comprises machine executable code that, upon execution by the one or more computer processors, implements any of the methods above or elsewhere herein.

In another aspect, described herein are construction materials comprising aggregate particles, calcium carbonate, and a supplemental material. In some embodiments, the mass ratio of the supplemental material to the calcium carbonate is no more than about 1:1. In some embodiments, the construction material has a compressive strength which is increased at least about 10% relative to an otherwise identical construction material wherein the supplemental material is not present.

In some embodiments, the construction material comprises aggregate particles, calcium carbonate, and about 0.1 weight % to about 40 weight % of a supplemental material. In some embodiments, the construction material has a compressive strength which is increased at least about 10% relative to an otherwise identical construction material wherein the supplemental material is not present. In some embodiments, the construction material has a compressive strength which is at least about 900 psi.

In some embodiments, the supplemental material is a metal sulfate. In some embodiments, the supplemental material is a metal silicate. In some embodiments, the supplemental material is a metal hydroxide. In some embodiments, the supplemental material is calcium sulfate. In some embodiments, the supplemental material is calcium hydroxide. In some embodiments, the supplemental material is calcium oxide. In some embodiments, wherein the supplemental material is a bentonite clay. In some embodiments, the supplemental material is a mixture comprising one or more components selected from the group of metal silicates, metal carbonates, metal sulfates, metal oxides, and metal hydroxides.

In some embodiments, the supplemental material is a mixture comprising calcium oxide, silica, alumina, iron oxide, magnesium oxide, and sulfites (e.g. Ordinary Portland cement). In some embodiments, the construction material further comprises fossilized cells of a microorganism. In some embodiments, the mass ratio of the supplemental material to the calcium carbonate is no more than about 0.2:1. In some embodiments, the calcium carbonate is from about 0.01 weight % to about 20 weight % of the construction material.

In some embodiments, a carbon footprint of the construction material is at least about 60% less than that of a functionally equivalent construction material made from ACI 318 structural concrete. In some embodiments, a carbon footprint of the construction material is at least about 80% less than that of a functionally equivalent construction material made from ACI 318 structural concrete. In some embodiments, a carbon footprint of the construction material is at least about 90% less than that of a functionally equivalent construction material made from ACI 318 structural concrete. In some embodiments, a carbon footprint of the construction material is at least about 95% less than that of a functionally equivalent construction material made from ACI 318 structural concrete.

In another aspect, described herein are methods of manufacturing a construction material described herein. In some embodiments, the method comprises combining aggregate particles with biocementation reagents and a supplemental material to yield a biocement mixture. In some embodiments, the method comprises reacting the cementation reagents in the presence of the supplemental material to produce a biocement, thereby yielding the construction material. In some embodiments, the steps of the method are carried out in a temperature-controlled and humidity-controlled environment.

In some embodiments, a temperature, humidity, and/or pH are monitored and controlled during the reacting. In some embodiments, the biocementation reagents comprise at least one biological organism or enzyme further comprising urease or cells of a urease-producing microorganism.

In some embodiments, the biocementation reagents comprise an acid-producing enzyme or cells of an acid-producing microorganism. In some embodiments, the biocementation reagents comprise carbonic anhydrase or cells of a carbonic anhydrase-producing microorganism. In some embodiments, the urease-producing microorganism is Sporosarcina pasteurii. In some embodiments, the urease-producing microorganism is selected from the group of: Sporosarcina pasteurii, Sporosarcina ureae, Proteus vulgaris, Bacillus sphaericus, Myxococcus xanthus, Proteus mirabilis, Bacillus megaterium, Helicobacter pylori, and combinations of two or more thereof. In some embodiments, the cells comprise spores. In some embodiments, the biocementation reagents comprise nutrients which promote the growth or enzymatic activities of microorganisms. In some embodiments, the nutrients comprise one or more of salts, amino acids, proteins, peptides, carbohydrates, saccharides, polysaccharides, fatty acids, oil, vitamins and minerals.

In some embodiments, the biocementation reagents comprise a calcium source. In some embodiments, the biocementation reagents comprise a urea source. In some embodiments, the biocementation reagents comprise calcium carbonate. In some embodiments, the biocementation reagents comprise calcium chloride. In some embodiments, the biocementation reagents comprise cells of a urea-producing microorganism. In some embodiments, the urea-producing microorganism is selected from the group of: Pseudomonas, Delaya avenusta, Thiosphaera pantotropha, Pseudomonas stutzen, Fragilaria crotonensis, Pseudoalteromonas spp., Pseudoalteromonas haloplanktis, Halomonas venusta, Pseudomonas balearica, Pseudomonas stutzeri, Bacillus megaterium, Exiguobacterium aurantiacum, Pseudoalteromonas aliena, Pseudoalteromonas luteoviolacea, E. coli, and variants, serotypes, mutations, recombinant forms, and combinations thereof. In some embodiments, the acid-producing microorganism is selected from the group consisting of: Variovorax, Klebsiella, Pseudomonas, Bacillus, Exiguobacterium, Microbacterium, Curtobacterium, Rathayibacter, CellFimi2, Streptomyces, Raoultella, B. pumilus, B. safanensis, B. simplex, B. licheniformis, and combinations thereof.

In some embodiments, the acid produced by the acid-producing enzyme of the cells of the acid-producing microorganism is a carboxylic acid. In some embodiments, the acid produced by the acid-producing enzyme of the cells of the acid-producing microorganism is acetic acid, formic acid, propionic acid, butyric acid, gluconic acid, succinic acid, lactic acid, or citric acid. In some embodiments, the method further comprises compacting the mixture to reduce the average linear distance between adjacent aggregate particles by at least about 25%. In some embodiments, the method further comprises compacting the mixture to produce an absolute packing efficiency of the aggregate particles of at least about 50%.

In some embodiments, the method further comprises compacting the mixture by placing the mixture in a vibratory press and applying pressure and vibration to reduce the volume of empty space between a plurality of biocement coated aggregate particles. In some embodiments, the vibratory motor of the press is operated at a rotational speed from about 100 RPM to about 7200 RPM. In some embodiments, the vibratory motor of the press is operated at a duty cycle from 0.01% to about 100%. In some embodiments, the vibration and pressure are applied simultaneously. In some embodiments, the vibration and pressure are applied alternatively. In some embodiments, the finished construction material comprises at least about 2% biocement by weight. In some embodiments, the finished construction material comprises at most about 20% biocement by weight.

In some embodiments, aggregate particles comprise natural, non-natural, recycled or manufactured sand, ore, crushed rock, stone, minerals, crushed or fractured glass, wood, ash, foam, basalt, fibers, mine tailings, paper, waste materials, waste from a manufacturing process, plastics, polymers, roughened materials, and/or combinations thereof. In some embodiments, the construction material comprises bricks, thin bricks, pavers, panels, tile, veneer, cinder, breeze, clinker or aerated blocks, counter-tops, table-tops, design structures, blocks, a solid masonry structure, piers, foundations, beams, walls, or slabs.

In some embodiments of the construction material or the methods described herein, the construction material comprises about 0.5 weight % to about 5 weight % of a supplemental material, and wherein the construction material has a compressive strength which is increased at least about 25% relative to an otherwise identical construction material wherein the supplemental material is not present. In some embodiments of the construction material or the methods described herein, the construction material comprises about 0.5% weight % to about 5 weight % of a supplemental material, and wherein the construction material has a compressive strength which is at least about 900 psi.

In some embodiments, any of the methods described herein can be used to produce any of the construction materials described herein. In some instances, any of the mixing devices described herein can be used in the performance of any of the methods described herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates an example workflow for producing a high-strength biocement material without hydroponics.

FIGS. 2A-D illustrates an example biocement mixing device.

FIG. 3 illustrates a diagram of an example controller for a biocement mixing device.

FIG. 4 illustrates an example of a press-formed bioconcrete material made by a method described herein.

FIG. 5 shows a computer system that is programmed or otherwise configured to implement methods provided herein.

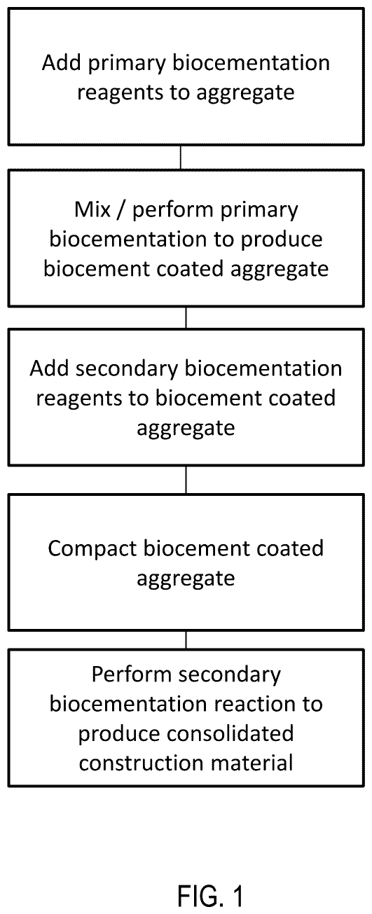

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain inventive embodiments herein contemplate numerical ranges. When ranges are present, the ranges include the range endpoints. Additionally, every sub range and value within the range is present as if explicitly written out. The term “about” or “approximately” may mean within an acceptable error range for the particular value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where particular values are described in the application and claims, unless otherwise stated the term “about” meaning within an acceptable error range for the particular value may be assumed.

As used herein, “biocement” generally refers to any binding agent generated through a biological mechanism (such as one or more enzymatic and/or metabolic processes) which adheres to or encapsulates particles of solid material. Examples of a biocement include calcium carbonate bound to a preexisting particle of a solid material which was formed by microbial induced calcium carbonate precipitation. Further examples include biologically sintered metal carbonates, such as, but not limited to calcium carbonate, magnesium carbonate, barium carbonate, or strontium carbonate.

As used herein, “biocement product” or “biocement products” are used to refer to an article which comprises elements or subcomponents that are bound together by biocement linkages or bridges. Examples of biocement products include but are not limited to items made from bioconcrete, biocement coated aggregates, and the like.

As used herein, “bioconcrete” is used to refer to a bulk composite material comprising biocement linkages or bridges which link together a network of aggregate particles.

As used herein, “aggregate” generally refers to any type of particulate matter which can be bound together into larger particles or consolidated solids by biocement bonds or bridges. Examples of particulates suitable for use as aggregate include sand, crushed stone, mine tailings, and similar particulate materials.

As used herein, “biocementation reagents” or “cementation reagents” generally refer to any combination of growth nutrients or starting materials, which when combined with an enzyme (or an organism containing an enzyme) lead to the enzymatic formation of a biocement. For example, an enzyme (or an organism containing an enzyme) can lead to the enzymatic formation of a biocement such as calcium carbonate which binds together adjacent aggregate particles. For example, cementation reagents in a urea-hydrolysis based biocementation system comprise urea (or another suitable nitrogen source), a soluble calcium source (e.g. calcium chloride, calcium acetate, calcium phosphate, calcium lactate, calcium nitrate, etc), and nutrients which promote urease activity (which will vary depending on whether pure enzyme or urease-producing cells are used) to form and precipitate a calcium carbonate biocement. In a calcium carbonate based biological sintering biocementation system, examples of biocementation reagents can comprise calcium carbonate, nutrients which promote enzymatic acid production, and an acid producing enzyme which generates acid to dissolve the calcium carbonate. Biocementation reagents for such a system may also comprise a second set of nutrients and a second enzyme which together promote a pH drop, reprecipitating calcium carbonate to form a biocement.

As used herein, “RPM” and “rpm” are used as units of an instantaneous rotational speed equivalent to revolutions per minute if that speed were maintained for a full minute (i.e. if the duty cycle is 100%).

As used herein, “duty cycle” refers to the percentage of the time a motor is on compared to when it is off. Duty cycle timing and waveform may be optimized for the specific step performed, however, unless otherwise specified duty cycle generally refers to a simple square waveform at a specified speed (i.e. motor on or motor off).

As used herein, “supplemental material” generally refers to a material added to a biocement composition that is different from an aggregate material which comprises a majority of the mass of a finished bioconcrete (e.g. greater than 50 weight % or a larger mass fraction than any other component) and which does not comprise biocementation reagents.

### Press-Formed Biocementation Processes

Described herein are scalable methods for producing high-strength bioconcrete construction materials without the need for hydroponics. In one aspect, an initial cementation reaction may be performed by adding measured doses of an enzyme or an enzyme-producing biological organism and biocementation reagents to aggregate particles. Either measured dose may be added in the form of an aqueous solution, a neat liquid, a solid, or combinations thereof as applicable for the individual biocementation reaction component employed. Water may optionally be added to control the humidity or to form an aqueous mixture when biocementation reactions and/or organisms are added in dry forms.

The aggregate material may comprise any type of natural rock or stone, glass, fiberglass, wood, biomass, paper, metal, plastic, polymers, rubber, imitation rubber, vinyl, minerals, imitations of rock or stone, recycled materials such as, for example, recycled brick, concrete, stone, mine tailings and mining residues, scrubber wastes, and/or combinations thereof. Aggregate can be of any size including mixtures of sizes provided such aggregate is smaller in size than the resulting structure. Aggregate materials may comprise particles of 10 mm or less, 5 mm or less, 1 mm or less, 0.5 mm or less, or combinations thereof. Mesh sizes can be as desired including but not limited to very fine particles (any number between and including 32-300 standard mesh), fine particles (any number between and including 10-32 standard mesh), medium particles (less than 10 standard mesh including all mesh numbers therein), and coarse particles (particles greater than or equal to 2 mm), and combinations thereof. Particle can be most any shape including, for example, round or rounded, oval, spherical (S grade), square, rectangular, hedral, fiber, lath, angular, elongated, needle-like, acicular, flat, flaky, cylindrical, spongy, cubic, cubical and combinations and variations thereof. If desired or necessary, aggregate particles can be roughened to create cracks in and crevices on particle surfaces.

Roughening can be performed by mixing particles together in a mixer or blender with sufficient force to create cracks in and crevices on particle surfaces, by adding ball bearings or another substance to the aggregate particles with a hardness equal to or greater than the particles themselves, by passing particles over a roughening agent such as, for example, sand, steel, or industrial diamonds, or another roughening agent known to those skilled in the art or combinations thereof.

The aggregate material may comprise rock (e.g., fines), sand, glass, wood, paper, metal, plastic, polymers, minerals, manufacturing or processing waste materials such as ash, carbon or wood residuals, any of which can be crushed or used whole or combinations thereof.

The aggregate may comprise organic or inorganic material such as, for example, sand, rock, glass (e.g. Poraver), wood, paper, metal, plastic, polymers, minerals, recycled materials, or combinations thereof. Aggregate particles may comprise beads, grains, rods, strands, fibers, flakes, crystals, pulverized or crushed materials, or combinations thereof.

Aggregate particles may have a diameter (e.g., actual, average or effective diameter) of about 50 mm or less, preferably about 25 mm or less, preferably about 20 mm or less, preferably about 10 mm or less, and preferably about 5 mm or less. In some embodiments, aggregate material may be about 1 mm or less and about 0.5 mm or less, about 0.1 mm or less, and about 50 μm or less. Particles sizes may include from about 10 μm to about 1 mm, from about 100 μm to about 0.5 mm, from about 200 μm to about 1 mm, from about 1 μm to about 200 μm, from about 10 nm to about 1 μm, and from about 10 nm to about 40 nm, and various combinations thereof. Aggregate particles may be largely composed of particulates of less than 12 mm in diameter (e.g. less than or about 12 mm, less than or about 8 mm, less than or about 5 mm, less than or about 4 mm, less than or about 3 mm, less than or about 2 mm, or less than or about 1 mm);

Aggregate particles may be characterized by fine size and may be equal to or less than 250 micron, equal to or less than 200 micron, equal to or less than 150 micron, or equal to or less than 100 micron (reference examples include micron size of beach sand=700, micron size of fine sand=250; micron size of Portland cement=74; micron size of silt=44; micron size of smoke=2).

In some embodiments large aggregate particles may be used, e.g. aggregates comprising particles of cm or mm size, e.g. gravels, stones, crushed rocks etc. In some embodiments, the size of the large aggregate particles is about 1 mm to 12 mm. In some embodiments, the size range of particles may range from 1 μm, or 10 μm, to 5 cm, or more. In some embodiments, the particulate starting material may consist of, or at least essentially consist of, small particles, in particular particles on a μm scale. In other words, the particles may be between, or in the range of 1 μm and 1 mm (1000 μm) in size, for instance between, or in the range of, 100 and 1000 μm or 100 and 500 μm, for example 200 and 400 μm, or 200 and 300 μm. Large size particles may however be, for instance, between 1 mm and 2 mm in size, or have a wider range in size, for instance between 100 μm and 2 mm.

In some embodiments, a maximum size of the aggregate particles is based on a thickness of the construction material produced by the method. The thickness of the construction material generally refers to the smallest dimension of the construction material. In some embodiments, the maximum size of the aggregate particles is a ratio of the thickness of the construction material.

In some embodiments, ratio of the maximum size of the aggregate particles to the thickness of the construction material is about 1 to 50, about 1 to 20, about 1 to 10, about 1 to 3, or about 1 to 2. In some embodiments, ratio of the maximum size of the aggregate particles to the thickness of the construction material is 1 to 50. In some embodiments, ratio of the maximum size of the aggregate particles to the thickness of the construction material is 1 to 20. In some embodiments, ratio of the maximum size of the aggregate particles to the thickness of the construction material is 1 to 10. In some embodiments, ratio of the maximum size of the aggregate particles to the thickness of the construction material is 1 to 3. In some embodiments, ratio of the maximum size of the aggregate particles to the thickness of the construction material is 1 to 2.

The initial cementation reaction may cause individual aggregate particles to conglomerate, yielding larger particles. The initial cementation reaction may coat individual aggregate particles with a biocement material to yield larger particles with or without conglomeration of the particles. The initial cementation reaction may form a biofilm on or around the individual aggregate particles. The biocement formed by the initial cementation reaction may adhere to individual aggregate particle surfaces, and may result in formation of some clustered particle-to-particle bonding. The material resulting from the initial cementation reaction may be amorphous in form and may comprise clusters or globules of semi-biocemented aggregate useful in downstream steps of methods described herein. The coated or enlarged aggregate particles may be stored for later use or may be used immediately to produce a bioconcrete construction material. The coated or enlarged aggregate particles may be stored indefinitely prior to downstream use.

A secondary cementation may be performed by adding a second dose of an enzyme or an enzyme-producing biological organism and/or a second dose of cementation reagents to the coated or enlarged aggregate particles produced by an initial cementation reaction. The enlarged or coated particles may be compacted, for example using a vibratory press, to reduce the space between individual aggregate particles prior to the completion of the secondary cementation reaction. Reducing the space between aggregate particles results in the formation of more biocement bonds between aggregate particles and/or stronger biocement bonds between individual aggregate particles, resulting in production of a stronger consolidated bioconcrete material by the method.

The biocement formed in the initial or secondary cementation reactions may bond to individual aggregate particles to create bridges between the particles or it may encapsulate individual particles. The enzyme or enzyme-producing biological organism and the biocementation reagents may be added together or separately in either the initial or the secondary cementation reagents. The biocement may be a precipitated calcium carbonate biocement. The enzyme producing biological organism may be a urease-producing microorganism.

The urease-producing microorganism may be selected from the group of: Sporosarcina pasteurii, Sporosarcina ureae, Proteus vulgaris, Bacillus sphaericus, Myxococcus xanthus, Proteus mirabilis, Bacillus megaterium, Helicobacter pylori, Rhodococcus erythropolis (B00038) and combinations of two or more thereof.

The enzyme producing biological organism may be an acid producing microorganism. The acid-producing microorganism may be selected from the group consisting of: Variovorax, Klebsiella, Pseudomonas, Bacillus, Exiguobacterium, Microbacterium, Curtobacterium, Rathayibacter, CellFimi2, Streptomyces, Raoultella, B. pumilus, B. safanensis, B. simplex, B. licheniformis, Lactobacillus, and combinations thereof.

Either cementation reaction may be mixed before, during, or after the addition of the enzyme, the biological organism, or the biocementation reagents. The mixing may be performed using a mixing device described herein. The temperature, humidity, and/or pH of each cementation reaction may each be independently controlled.

Each individual step of the method may independently be performed in about 0.1 hours to about 72 hours. Each individual step of the method may independently be performed in about 0.1 hours to about 1 hour, about 0.1 hours to about 2 hours, about 0.1 hours to about 4 hours, about 0.1 hours to about 6 hours, about 0.1 hours to about 8 hours, about 0.1 hours to about 12 hours, about 0.1 hours to about 16 hours, about 0.1 hours to about 24 hours, about 0.1 hours to about 36 hours, about 0.1 hours to about 48 hours, about 0.1 hours to about 72 hours, about 1 hour to about 2 hours, about 1 hour to about 4 hours, about 1 hour to about 6 hours, about 1 hour to about 8 hours, about 1 hour to about 12 hours, about 1 hour to about 16 hours, about 1 hour to about 24 hours, about 1 hour to about 36 hours, about 1 hour to about 48 hours, about 1 hour to about 72 hours, about 2 hours to about 4 hours, about 2 hours to about 6 hours, about 2 hours to about 8 hours, about 2 hours to about 12 hours, about 2 hours to about 16 hours, about 2 hours to about 24 hours, about 2 hours to about 36 hours, about 2 hours to about 48 hours, about 2 hours to about 72 hours, about 4 hours to about 6 hours, about 4 hours to about 8 hours, about 4 hours to about 12 hours, about 4 hours to about 16 hours, about 4 hours to about 24 hours, about 4 hours to about 36 hours, about 4 hours to about 48 hours, about 4 hours to about 72 hours, about 6 hours to about 8 hours, about 6 hours to about 12 hours, about 6 hours to about 16 hours, about 6 hours to about 24 hours, about 6 hours to about 36 hours, about 6 hours to about 48 hours, about 6 hours to about 72 hours, about 8 hours to about 12 hours, about 8 hours to about 16 hours, about 8 hours to about 24 hours, about 8 hours to about 36 hours, about 8 hours to about 48 hours, about 8 hours to about 72 hours, about 12 hours to about 16 hours, about 12 hours to about 24 hours, about 12 hours to about 36 hours, about 12 hours to about 48 hours, about 12 hours to about 72 hours, about 16 hours to about 24 hours, about 16 hours to about 36 hours, about 16 hours to about 48 hours, about 16 hours to about 72 hours, about 24 hours to about 36 hours, about 24 hours to about 48 hours, about 24 hours to about 72 hours, about 36 hours to about 48 hours, about 36 hours to about 72 hours, or about 48 hours to about 72 hours. Each individual step of the method may independently be performed in about 0.1 hours, about 1 hour, about 2 hours, about 4 hours, about 6 hours, about 8 hours, about 12 hours, about 16 hours, about 24 hours, about 36 hours, about 48 hours, or about 72 hours. Each individual step of the method may independently be performed in at least about 0.1 hours, about 1 hour, about 2 hours, about 4 hours, about 6 hours, about 8 hours, about 12 hours, about 16 hours, about 24 hours, about 36 hours, or about 48 hours. Each individual step of the method may independently be performed in at most about 1 hour, about 2 hours, about 4 hours, about 6 hours, about 8 hours, about 12 hours, about 16 hours, about 24 hours, about 36 hours, about 48 hours, or about 72 hours.

The temperature of each step of the method may be controlled to be about 20° C. to about 45° C. The temperature of each step of the method may be controlled to be about 20° C. to about 22° C., about 20° C. to about 24° C., about 20° C. to about 26° C., about 20° C. to about 28° C., about 20° C. to about 30° C., about 20° C. to about 32° C., about 20° C. to about 34° C., about 20° C. to about 36° C., about 20° C. to about 38° C., about 20° C. to about 40° C., about 20° C. to about 45° C., about 22° C. to about 24° C., about 22° C. to about 26° C., about 22° C. to about 28° C., about 22° C. to about 30° C., about 22° C. to about 32° C., about 22° C. to about 34° C., about 22° C. to about 36° C., about 22° C. to about 38° C., about 22° C. to about 40° C., about 22° C. to about 45° C., about 24° C. to about 26° C., about 24° C. to about 28° C., about 24° C. to about 30° C., about 24° C. to about 32° C., about 24° C. to about 34° C., about 24° C. to about 36° C., about 24° C. to about 38° C., about 24° C. to about 40° C., about 24° C. to about 45° C., about 26° C. to about 28° C., about 26° C. to about 30° C., about 26° C. to about 32° C., about 26° C. to about 34° C., about 26° C. to about 36° C., about 26° C. to about 38° C., about 26° C. to about 40° C., about 26° C. to about 45° C., about 28° C. to about 30° C., about 28° C. to about 32° C., about 28° C. to about 34° C., about 28° C. to about 36° C., about 28° C. to about 38° C., about 28° C. to about 40° C., about 28° C. to about 45° C., about 30° C. to about 32° C., about 30° C. to about 34° C., about 30° C. to about 36° C., about 30° C. to about 38° C., about 30° C. to about 40° C., about 30° C. to about 45° C., about 32° C. to about 34° C., about 32° C. to about 36° C., about 32° C. to about 38° C., about 32° C. to about 40° C., about 32° C. to about 45° C., about 34° C. to about 36° C., about 34° C. to about 38° C., about 34° C. to about 40° C., about 34° C. to about 45° C., about 36° C. to about 38° C., about 36° C. to about 40° C., about 36° C. to about 45° C., about 38° C. to about 40° C., about 38° C. to about 45° C., or about 40° C. to about 45° C. The temperature of each step of the method may be controlled to be about 20° C., about 22° C., about 24° C., about 26° C., about 28° C., about 30° C., about 32° C., about 34° C., about 36° C., about 38° C., about 40° C., or about 45° C. The temperature of each step of the method may be controlled to be at least about 20° C., about 22° C., about 24° C., about 26° C., about 28° C., about 30° C., about 32° C., about 34° C., about 36° C., about 38° C., or about 40° C. The temperature of each step of the method may be controlled to be at most about 22° C., about 24° C., about 26° C., about 28° C., about 30° C., about 32° C., about 34° C., about 36° C., about 38° C., about 40° C., or about 45° C.

The total amount of solution added during either of each cementation reaction may be about 0.5 L/kg of aggregate to about 50 L/kg of aggregate. The total amount of solution added during either of each cementation reaction may be about 0.5 L/kg of aggregate to about 1 L/kg of aggregate, about 0.5 L/kg of aggregate to about 2 L/kg of aggregate, about 0.5 L/kg of aggregate to about 5 L/kg of aggregate, about 0.5 L/kg of aggregate to about 7 L/kg of aggregate, about 0.5 L/kg of aggregate to about 10 L/kg of aggregate, about 0.5 L/kg of aggregate to about 12 L/kg of aggregate, about 0.5 L/kg of aggregate to about 14 L/kg of aggregate, about 0.5 L/kg of aggregate to about 18 L/kg of aggregate, about 0.5 L/kg of aggregate to about 36 L/kg of aggregate, about 0.5 L/kg of aggregate to about 42 L/kg of aggregate, about 0.5 L/kg of aggregate to about 50 L/kg of aggregate, about 1 L/kg of aggregate to about 2 L/kg of aggregate, about 1 L/kg of aggregate to about 5 L/kg of aggregate, about 1 L/kg of aggregate to about 7 L/kg of aggregate, about 1 L/kg of aggregate to about 10 L/kg of aggregate, about 1 L/kg of aggregate to about 12 L/kg of aggregate, about 1 L/kg of aggregate to about 14 L/kg of aggregate, about 1 L/kg of aggregate to about 18 L/kg of aggregate, about 1 L/kg of aggregate to about 36 L/kg of aggregate, about 1 L/kg of aggregate to about 42 L/kg of aggregate, about 1 L/kg of aggregate to about 50 L/kg of aggregate, about 2 L/kg of aggregate to about 5 L/kg of aggregate, about 2 L/kg of aggregate to about 7 L/kg of aggregate, about 2 L/kg of aggregate to about 10 L/kg of aggregate, about 2 L/kg of aggregate to about 12 L/kg of aggregate, about 2 L/kg of aggregate to about 14 L/kg of aggregate, about 2 L/kg of aggregate to about 18 L/kg of aggregate, about 2 L/kg of aggregate to about 36 L/kg of aggregate, about 2 L/kg of aggregate to about 42 L/kg of aggregate, about 2 L/kg of aggregate to about 50 L/kg of aggregate, about 5 L/kg of aggregate to about 7 L/kg of aggregate, about 5 L/kg of aggregate to about 10 L/kg of aggregate, about 5 L/kg of aggregate to about 12 L/kg of aggregate, about 5 L/kg of aggregate to about 14 L/kg of aggregate, about 5 L/kg of aggregate to about 18 L/kg of aggregate, about 5 L/kg of aggregate to about 36 L/kg of aggregate, about 5 L/kg of aggregate to about 42 L/kg of aggregate, about 5 L/kg of aggregate to about 50 L/kg of aggregate, about 7 L/kg of aggregate to about 10 L/kg of aggregate, about 7 L/kg of aggregate to about 12 L/kg of aggregate, about 7 L/kg of aggregate to about 14 L/kg of aggregate, about 7 L/kg of aggregate to about 18 L/kg of aggregate, about 7 L/kg of aggregate to about 36 L/kg of aggregate, about 7 L/kg of aggregate to about 42 L/kg of aggregate, about 7 L/kg of aggregate to about 50 L/kg of aggregate, about 10 L/kg of aggregate to about 12 L/kg of aggregate, about 10 L/kg of aggregate to about 14 L/kg of aggregate, about 10 L/kg of aggregate to about 18 L/kg of aggregate, about 10 L/kg of aggregate to about 36 L/kg of aggregate, about 10 L/kg of aggregate to about 42 L/kg of aggregate, about 10 L/kg of aggregate to about 50 L/kg of aggregate, about 12 L/kg of aggregate to about 14 L/kg of aggregate, about 12 L/kg of aggregate to about 18 L/kg of aggregate, about 12 L/kg of aggregate to about 36 L/kg of aggregate, about 12 L/kg of aggregate to about 42 L/kg of aggregate, about 12 L/kg of aggregate to about 50 L/kg of aggregate, about 14 L/kg of aggregate to about 18 L/kg of aggregate, about 14 L/kg of aggregate to about 36 L/kg of aggregate, about 14 L/kg of aggregate to about 42 L/kg of aggregate, about 14 L/kg of aggregate to about 50 L/kg of aggregate, about 18 L/kg of aggregate to about 36 L/kg of aggregate, about 18 L/kg of aggregate to about 42 L/kg of aggregate, about 18 L/kg of aggregate to about 50 L/kg of aggregate, about 36 L/kg of aggregate to about 42 L/kg of aggregate, about 36 L/kg of aggregate to about 50 L/kg of aggregate, or about 42 L/kg of aggregate to about 50 L/kg of aggregate. The total amount of solution added during either of each cementation reaction may be about 0.5 L/kg of aggregate, about 1 L/kg of aggregate, about 2 L/kg of aggregate, about 5 L/kg of aggregate, about 7 L/kg of aggregate, about 10 L/kg of aggregate, about 12 L/kg of aggregate, about 14 L/kg of aggregate, about 18 L/kg of aggregate, about 36 L/kg of aggregate, about 42 L/kg of aggregate, or about 50 L/kg of aggregate. The total amount of solution added during either of each cementation reaction may be at least about 0.5 L/kg of aggregate, about 1 L/kg of aggregate, about 2 L/kg of aggregate, about 5 L/kg of aggregate, about 7 L/kg of aggregate, about 10 L/kg of aggregate, about 12 L/kg of aggregate, about 14 L/kg of aggregate, about 18 L/kg of aggregate, about 36 L/kg of aggregate, or about 42 L/kg of aggregate. The total amount of solution added during either of each cementation reaction may be at most about 1 L/kg of aggregate, about 2 L/kg of aggregate, about 5 L/kg of aggregate, about 7 L/kg of aggregate, about 10 L/kg of aggregate, about 12 L/kg of aggregate, about 14 L/kg of aggregate, about 18 L/kg of aggregate, about 36 L/kg of aggregate, about 42 L/kg of aggregate, or about 50 L/kg of aggregate.

The concentration of calcium ions in either cementation reagent solution may independently be about 0.1 M to about 3 M. The concentration of calcium ions in either cementation reagent solution may independently be about 0.1 M to about 0.2 M, about 0.1 M to about 0.3 M, about 0.1 M to about 0.4 M, about 0.1 M to about 0.5 M, about 0.1 M to about 0.7 M, about 0.1 M to about 0.9 M, about 0.1 M to about 1 M, about 0.1 M to about 1.2 M, about 0.1 M to about 1.5 M, about 0.1 M to about 2 M, about 0.1 M to about 3 M, about 0.2 M to about 0.3 M, about 0.2 M to about 0.4 M, about 0.2 M to about 0.5 M, about 0.2 M to about 0.7 M, about 0.2 M to about 0.9 M, about 0.2 M to about 1 M, about 0.2 M to about 1.2 M, about 0.2 M to about 1.5 M, about 0.2 M to about 2 M, about 0.2 M to about 3 M, about 0.3 M to about 0.4 M, about 0.3 M to about 0.5 M, about 0.3 M to about 0.7 M, about 0.3 M to about 0.9 M, about 0.3 M to about 1 M, about 0.3 M to about 1.2 M, about 0.3 M to about 1.5 M, about 0.3 M to about 2 M, about 0.3 M to about 3 M, about 0.4 M to about 0.5 M, about 0.4 M to about 0.7 M, about 0.4 M to about 0.9 M, about 0.4 M to about 1 M, about 0.4 M to about 1.2 M, about 0.4 M to about 1.5 M, about 0.4 M to about 2 M, about 0.4 M to about 3 M, about 0.5 M to about 0.7 M, about 0.5 M to about 0.9 M, about 0.5 M to about 1 M, about 0.5 M to about 1.2 M, about 0.5 M to about 1.5 M, about 0.5 M to about 2 M, about 0.5 M to about 3 M, about 0.7 M to about 0.9 M, about 0.7 M to about 1 M, about 0.7 M to about 1.2 M, about 0.7 M to about 1.5 M, about 0.7 M to about 2 M, about 0.7 M to about 3 M, about 0.9 M to about 1 M, about 0.9 M to about 1.2 M, about 0.9 M to about 1.5 M, about 0.9 M to about 2 M, about 0.9 M to about 3 M, about 1 M to about 1.2 M, about 1 M to about 1.5 M, about 1 M to about 2 M, about 1 M to about 3 M, about 1.2 M to about 1.5 M, about 1.2 M to about 2 M, about 1.2 M to about 3 M, about 1.5 M to about 2 M, about 1.5 M to about 3 M, or about 2 M to about 3 M. The concentration of calcium ions in either cementation reagent solution may independently be about 0.1 M, about 0.2 M, about 0.3 M, about 0.4 M, about 0.5 M, about 0.7 M, about 0.9 M, about 1 M, about 1.2 M, about 1.5 M, about 2 M, or about 3 M. The concentration of calcium ions in either cementation reagent solution may independently be at least about 0.1 M, about 0.2 M, about 0.3 M, about 0.4 M, about 0.5 M, about 0.7 M, about 0.9 M, about 1 M, about 1.2 M, about 1.5 M, or about 2 M. The concentration of calcium ions in either cementation reagent solution may independently be at most about 0.2 M, about 0.3 M, about 0.4 M, about 0.5 M, about 0.7 M, about 0.9 M, about 1 M, about 1.2 M, about 1.5 M, about 2 M, or about 3 M. The concentration of urea in either cementation reagent solution may independently be about 0.1 M to about 3 M. The concentration of urea in either cementation reagent solution may independently be about 0.1 M to about 0.2 M, about 0.1 M to about 0.3 M, about 0.1 M to about 0.4 M, about 0.1 M to about 0.5 M, about 0.1 M to about 0.7 M, about 0.1 M to about 0.9 M, about 0.1 M to about 1 M, about 0.1 M to about 1.2 M, about 0.1 M to about 1.5 M, about 0.1 M to about 2 M, about 0.1 M to about 3 M, about 0.2 M to about 0.3 M, about 0.2 M to about 0.4 M, about 0.2 M to about 0.5 M, about 0.2 M to about 0.7 M, about 0.2 M to about 0.9 M, about 0.2 M to about 1 M, about 0.2 M to about 1.2 M, about 0.2 M to about 1.5 M, about 0.2 M to about 2 M, about 0.2 M to about 3 M, about 0.3 M to about 0.4 M, about 0.3 M to about 0.5 M, about 0.3 M to about 0.7 M, about 0.3 M to about 0.9 M, about 0.3 M to about 1 M, about 0.3 M to about 1.2 M, about 0.3 M to about 1.5 M, about 0.3 M to about 2 M, about 0.3 M to about 3 M, about 0.4 M to about 0.5 M, about 0.4 M to about 0.7 M, about 0.4 M to about 0.9 M, about 0.4 M to about 1 M, about 0.4 M to about 1.2 M, about 0.4 M to about 1.5 M, about 0.4 M to about 2 M, about 0.4 M to about 3 M, about 0.5 M to about 0.7 M, about 0.5 M to about 0.9 M, about 0.5 M to about 1 M, about 0.5 M to about 1.2 M, about 0.5 M to about 1.5 M, about 0.5 M to about 2 M, about 0.5 M to about 3 M, about 0.7 M to about 0.9 M, about 0.7 M to about 1 M, about 0.7 M to about 1.2 M, about 0.7 M to about 1.5 M, about 0.7 M to about 2 M, about 0.7 M to about 3 M, about 0.9 M to about 1 M, about 0.9 M to about 1.2 M, about 0.9 M to about 1.5 M, about 0.9 M to about 2 M, about 0.9 M to about 3 M, about 1 M to about 1.2 M, about 1 M to about 1.5 M, about 1 M to about 2 M, about 1 M to about 3 M, about 1.2 M to about 1.5 M, about 1.2 M to about 2 M, about 1.2 M to about 3 M, about 1.5 M to about 2 M, about 1.5 M to about 3 M, or about 2 M to about 3 M. The concentration of urea in either cementation reagent solution may independently be about 0.1 M, about 0.2 M, about 0.3 M, about 0.4 M, about 0.5 M, about 0.7 M, about 0.9 M, about 1 M, about 1.2 M, about 1.5 M, about 2 M, or about 3 M. The concentration of urea in either cementation reagent solution may independently be at least about 0.1 M, about 0.2 M, about 0.3 M, about 0.4 M, about 0.5 M, about 0.7 M, about 0.9 M, about 1 M, about 1.2 M, about 1.5 M, or about 2 M. The concentration of urea in either cementation reagent solution may independently be at most about 0.2 M, about 0.3 M, about 0.4 M, about 0.5 M, about 0.7 M, about 0.9 M, about 1 M, about 1.2 M, about 1.5 M, about 2 M, or about 3 M.

The moisture level of the mixture during either cementation reaction may be controlled to be about 1 weight % to about 25 weight %. The moisture level of the mixture during either cementation reaction may be controlled to be about 1 weight % to about 2 weight %, about 1 weight % to about 3 weight %, about 1 weight % to about 4 weight %, about 1 weight % to about 5 weight %, about 1 weight % to about 7 weight %, about 1 weight % to about 9 weight %, about 1 weight % to about 10 weight %, about 1 weight % to about 12 weight %, about 1 weight % to about 14 weight %, about 1 weight % to about 15 weight %, about 1 weight % to about 25 weight %, about 2 weight % to about 3 weight %, about 2 weight % to about 4 weight %, about 2 weight % to about 5 weight %, about 2 weight % to about 7 weight %, about 2 weight % to about 9 weight %, about 2 weight % to about 10 weight %, about 2 weight % to about 12 weight %, about 2 weight % to about 14 weight %, about 2 weight % to about 15 weight %, about 2 weight % to about 25 weight %, about 3 weight % to about 4 weight %, about 3 weight % to about 5 weight %, about 3 weight % to about 7 weight %, about 3 weight % to about 9 weight %, about 3 weight % to about 10 weight %, about 3 weight % to about 12 weight %, about 3 weight % to about 14 weight %, about 3 weight % to about 15 weight %, about 3 weight % to about 25 weight %, about 4 weight % to about 5 weight %, about 4 weight % to about 7 weight %, about 4 weight % to about 9 weight %, about 4 weight % to about 10 weight %, about 4 weight % to about 12 weight %, about 4 weight % to about 14 weight %, about 4 weight % to about 15 weight %, about 4 weight % to about 25 weight %, about 5 weight % to about 7 weight %, about 5 weight % to about 9 weight %, about 5 weight % to about 10 weight %, about 5 weight % to about 12 weight %, about 5 weight % to about 14 weight %, about 5 weight % to about 15 weight %, about 5 weight % to about 25 weight %, about 7 weight % to about 9 weight %, about 7 weight % to about 10 weight %, about 7 weight % to about 12 weight %, about 7 weight % to about 14 weight %, about 7 weight % to about 15 weight %, about 7 weight % to about 25 weight %, about 9 weight % to about 10 weight %, about 9 weight % to about 12 weight %, about 9 weight % to about 14 weight %, about 9 weight % to about 15 weight %, about 9 weight % to about 25 weight %, about 10 weight % to about 12 weight %, about 10 weight % to about 14 weight %, about 10 weight % to about 15 weight %, about 10 weight % to about 25 weight %, about 12 weight % to about 14 weight %, about 12 weight % to about 15 weight %, about 12 weight % to about 25 weight %, about 14 weight % to about 15 weight %, about 14 weight % to about 25 weight %, or about 15 weight % to about 25 weight %. The moisture level of the mixture during either cementation reaction may be controlled to be about 1 weight %, about 2 weight %, about 3 weight %, about 4 weight %, about 5 weight %, about 7 weight %, about 9 weight %, about 10 weight %, about 12 weight 9%, about 14 weight %, about 15 weight %, or about 25 weight %. The moisture level of the mixture during either cementation reaction may be controlled to be at least about 1 weight %, about 2 weight %, about 3 weight %, about 4 weight %, about 5 weight %, about 7 weight %, about 9 weight %, about 10 weight %, about 12 weight %, about 14 weight %, or about 15 weight %. The moisture level of the mixture during either cementation reaction may be controlled to be at most about 2 weight %, about 3 weight %, about 4 weight %, about 5 weight %, about 7 weight %, about 9 weight %, about 10 weight %, about 12 weight %, about 14 weight %, about 15 weight %, or about 25 weight %.

In some embodiments, the finished bioconcrete construction material comprises about 0.5 weight % biocement to about 25 weight % biocement. In some embodiments, the finished bioconcrete construction material comprises about 0.5 weight % biocement to about 1 weight % biocement, about 0.5 weight % biocement to about 2 weight % biocement, about 0.5 weight % biocement to about 3 weight % biocement, about 0.5 weight % biocement to about 5 weight % biocement, about 0.5 weight % biocement to about 7 weight % biocement, about 0.5 weight % biocement to about 9 weight % biocement, about 0.5 weight % biocement to about 10 weight % biocement, about 0.5 weight % biocement to about 12 weight % biocement, about 0.5 weight % biocement to about 16 weight % biocement, about 0.5 weight % biocement to about 18 weight % biocement, about 0.5 weight % biocement to about 25 weight % biocement, about 1 weight % biocement to about 2 weight % biocement, about 1 weight % biocement to about 3 weight % biocement, about 1 weight % biocement to about 5 weight % biocement, about 1 weight % biocement to about 7 weight % biocement, about 1 weight % biocement to about 9 weight % biocement, about 1 weight % biocement to about 10 weight % biocement, about 1 weight % biocement to about 12 weight % biocement, about 1 weight % biocement to about 16 weight % biocement, about 1 weight % biocement to about 18 weight % biocement, about 1 weight % biocement to about 25 weight % biocement, about 2 weight % biocement to about 3 weight % biocement, about 2 weight % biocement to about 5 weight % biocement, about 2 weight % biocement to about 7 weight % biocement, about 2 weight % biocement to about 9 weight % biocement, about 2 weight % biocement to about 10 weight % biocement, about 2 weight % biocement to about 12 weight % biocement, about 2 weight % biocement to about 16 weight % biocement, about 2 weight % biocement to about 18 weight % biocement, about 2 weight % biocement to about 25 weight % biocement, about 3 weight % biocement to about 5 weight % biocement, about 3 weight % biocement to about 7 weight % biocement, about 3 weight % biocement to about 9 weight % biocement, about 3 weight % biocement to about 10 weight % biocement, about 3 weight % biocement to about 12 weight % biocement, about 3 weight % biocement to about 16 weight % biocement, about 3 weight % biocement to about 18 weight % biocement, about 3 weight % biocement to about 25 weight % biocement, about 5 weight % biocement to about 7 weight % biocement, about 5 weight % biocement to about 9 weight % biocement, about 5 weight % biocement to about 10 weight % biocement, about 5 weight % biocement to about 12 weight % biocement, about 5 weight % biocement to about 16 weight % biocement, about 5 weight % biocement to about 18 weight % biocement, about 5 weight % biocement to about 25 weight % biocement, about 7 weight % biocement to about 9 weight % biocement, about 7 weight % biocement to about 10 weight % biocement, about 7 weight % biocement to about 12 weight % biocement, about 7 weight % biocement to about 16 weight % biocement, about 7 weight % biocement to about 18 weight % biocement, about 7 weight % biocement to about 25 weight % biocement, about 9 weight % biocement to about 10 weight % biocement, about 9 weight % biocement to about 12 weight % biocement, about 9 weight % biocement to about 16 weight % biocement, about 9 weight % biocement to about 18 weight % biocement, about 9 weight % biocement to about 25 weight % biocement, about 10 weight % biocement to about 12 weight % biocement, about 10 weight % biocement to about 16 weight % biocement, about 10 weight % biocement to about 18 weight % biocement, about 10 weight % biocement to about 25 weight % biocement, about 12 weight % biocement to about 16 weight % biocement, about 12 weight % biocement to about 18 weight % biocement, about 12 weight % biocement to about 25 weight % biocement, about 16 weight % biocement to about 18 weight % biocement, about 16 weight % biocement to about 25 weight % biocement, or about 18 weight % biocement to about 25 weight % biocement. In some embodiments, the finished bioconcrete construction material comprises about 0.5 weight % biocement, about 1 weight % biocement, about 2 weight % biocement, about 3 weight % biocement, about 5 weight % biocement, about 7 weight % biocement, about 9 weight % biocement, about 10 weight % biocement, about 12 weight % biocement, about 16 weight % biocement, about 18 weight % biocement, or about 25 weight % biocement. In some embodiments, the finished bioconcrete construction material comprises at least about 0.5 weight % biocement, about 1 weight % biocement, about 2 weight % biocement, about 3 weight % biocement, about 5 weight % biocement, about 7 weight % biocement, about 9 weight % biocement, about 10 weight % biocement, about 12 weight % biocement, about 16 weight % biocement, or about 18 weight % biocement. In some embodiments, the finished bioconcrete construction material comprises at most about 1 weight % biocement, about 2 weight % biocement, about 3 weight % biocement, about 5 weight % biocement, about 7 weight % biocement, about 9 weight % biocement, about 10 weight % biocement, about 12 weight % biocement, about 16 weight % biocement, about 18 weight % biocement, or about 25 weight % biocement.

In some embodiments, the vibratory press is operated at a frequency of about 1 Hz to about 120 Hz. In some embodiments, the vibratory press is operated at a frequency of about 1 Hz to about 5 Hz, about 1 Hz to about 10 Hz, about 1 Hz to about 20 Hz, about 1 Hz to about 30 Hz, about 1 Hz to about 40 Hz, about 1 Hz to about 47 Hz, about 1 Hz to about 60 Hz, about 1 Hz to about 70 Hz, about 1 Hz to about 80 Hz, about 1 Hz to about 100 Hz, about 1 Hz to about 120 Hz, about 5 Hz to about 10 Hz, about 5 Hz to about 20 Hz, about 5 Hz to about 30 Hz, about 5 Hz to about 40 Hz, about 5 Hz to about 47 Hz, about 5 Hz to about 60 Hz, about 5 Hz to about 70 Hz, about 5 Hz to about 80 Hz, about 5 Hz to about 100 Hz, about 5 Hz to about 120 Hz, about 10 Hz to about 20 Hz, about 10 Hz to about 30 Hz, about 10 Hz to about 40 Hz, about 10 Hz to about 47 Hz, about 10 Hz to about 60 Hz, about 10 Hz to about 70 Hz, about 10 Hz to about 80 Hz, about 10 Hz to about 100 Hz, about 10 Hz to about 120 Hz, about 20 Hz to about 30 Hz, about 20 Hz to about 40 Hz, about 20 Hz to about 47 Hz, about 20 Hz to about 60 Hz, about 20 Hz to about 70 Hz, about 20 Hz to about 80 Hz, about 20 Hz to about 100 Hz, about 20 Hz to about 120 Hz, about 30 Hz to about 40 Hz, about 30 Hz to about 47 Hz, about 30 Hz to about 60 Hz, about 30 Hz to about 70 Hz, about 30 Hz to about 80 Hz, about 30 Hz to about 100 Hz, about 30 Hz to about 120 Hz, about 40 Hz to about 47 Hz, about 40 Hz to about 60 Hz, about 40 Hz to about 70 Hz, about 40 Hz to about 80 Hz, about 40 Hz to about 100 Hz, about 40 Hz to about 120 Hz, about 47 Hz to about 60 Hz, about 47 Hz to about 70 Hz, about 47 Hz to about 80 Hz, about 47 Hz to about 100 Hz, about 47 Hz to about 120 Hz, about 60 Hz to about 70 Hz, about 60 Hz to about 80 Hz, about 60 Hz to about 100 Hz, about 60 Hz to about 120 Hz, about 70 Hz to about 80 Hz, about 70 Hz to about 100 Hz, about 70 Hz to about 120 Hz, about 80 Hz to about 100 Hz, about 80 Hz to about 120 Hz, or about 100 Hz to about 120 Hz. In some embodiments, the vibratory press is operated at a frequency of about 1 Hz, about 5 Hz, about 10 Hz, about 20 Hz, about 30 Hz, about 40 Hz, about 47 Hz, about 60 Hz, about 70 Hz, about 80 Hz, about 100 Hz, or about 120 Hz. In some embodiments, the vibratory press is operated at a frequency of at least about 1 Hz, about 5 Hz, about 10 Hz, about 20 Hz, about 30 Hz, about 40 Hz, about 47 Hz, about 60 Hz, about 70 Hz, about 80 Hz, or about 100 Hz. In some embodiments, the vibratory press is operated at a frequency of at most about 5 Hz, about 10 Hz, about 20 Hz, about 30 Hz, about 40 Hz, about 47 Hz, about 60 Hz, about 70 Hz, about 80 Hz, about 100 Hz, or about 120 Hz.

In some embodiments, compacting the mixture results in an absolute packing efficiency of the coated or enlarged aggregate particles of about 25% to about 80%. In some embodiments, compacting the mixture results in an absolute packing efficiency of the coated or enlarged aggregate particles of about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 25% to about 80%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 30% to about 80%, about 35% to about 40%, about 35% to about 45%, about 35% to about 50%, about 35% to about 55%, about 35% to about 60%, about 35% to about 65%, about 35% to about 70%, about 35% to about 75%, about 35% to about 80%, about 40% to about 45%, about 40% to about 50%, about 40% to about 55%, about 40% to about 60%, about 40% to about 65%, about 40% to about 70%, about 40% to about 75%, about 40% to about 80%, about 45% to about 50%, about 45% to about 55%, about 45% to about 60%, about 45% to about 65%, about 45% to about 70%, about 45% to about 75%, about 45% to about 80%, about 50% to about 55%, about 50% to about 60%, about 50% to about 65%, about 50% to about 70%, about 50% to about 75%, about 50% to about 80%, about 55% to about 60%, about 55% to about 65%, about 55% to about 70%, about 55% to about 75%, about 55% to about 80%, about 60% to about 65%, about 60% to about 70%, about 60% to about 75%, about 60% to about 80%, about 65% to about 70%, about 65% to about 75%, about 65% to about 80%, about 70% to about 75%, about 70% to about 80%, or about 75% to about 80%. In some embodiments, compacting the mixture results in an absolute packing efficiency of the coated or enlarged aggregate particles of about 25%, about 30%, about 35%, about 40%, about 45%, about 50%, about 55%, about 60%, about 65%, about 70%, about 75%, or about 80%. In some embodiments, compacting the mixture results in an absolute packing efficiency of the coated or enlarged aggregate particles of at least about 25%, about 30%, about 35%, about 40%, about 45%, about 50%, about 55%, about 60%, about 65%, about 70%, or about 75%. In some embodiments, compacting the mixture results in an absolute packing efficiency of the coated or enlarged aggregate particles of at most about 30%, about 35%, about 40%, about 45%, about 50%, about 55%, about 60%, about 65%, about 70%, about 75%, or about 80%.

In some embodiments, compacting the mixture results in a relative compacted volume of the coated or enlarged aggregate particles compared to the volume prior to compaction of about 40% to about 95%. In some embodiments, compacting the mixture results in a relative compacted volume of the coated or enlarged aggregate particles compared to the volume prior to compaction of about 40% to about 50%, about 40% to about 55%, about 40% to about 60%, about 40% to about 65%, about 40% to about 70%, about 40% to about 75%, about 40% to about 80%, about 40% to about 85%, about 40% to about 90%, about 40% to about 95%, about 50% to about 55%, about 50% to about 60%, about 50% to about 65%, about 50% to about 70%, about 50% to about 75%, about 50% to about 80%, about 50% to about 85%, about 50% to about 90%, about 50% to about 95%, about 55% to about 60%, about 55% to about 65%, about 55% to about 70%, about 55% to about 75%, about 55% to about 80%, about 55% to about 85%, about 55% to about 90%, about 55% to about 95%, about 60% to about 65%, about 60% to about 70%, about 60% to about 75%, about 60% to about 80%, about 60% to about 85%, about 60% to about 90%, about 60% to about 95%, about 65% to about 70%, about 65% to about 75%, about 65% to about 80%, about 65% to about 85%, about 65% to about 90%, about 65% to about 95%, about 70% to about 75%, about 70% to about 80%, about 70% to about 85%, about 70% to about 90%, about 70% to about 95%, about 75% to about 80%, about 75% to about 85%, about 75% to about 90%, about 75% to about 95%, about 80% to about 85%, about 80% to about 90%, about 80% to about 95%, about 85% to about 90%, about 85% to about 95%, or about 90% to about 95%. In some embodiments, compacting the mixture results in a relative compacted volume of the coated or enlarged aggregate particles compared to the volume prior to compaction of about 40%, about 50%, about 55%, about 60%, about 65%, about 70%, about 75%, about 80%, about 85%, about 90%, or about 95%. In some embodiments, compacting the mixture results in a relative compacted volume of the coated or enlarged aggregate particles compared to the volume prior to compaction of at least about 40%, about 50%, about 55%, about 60%, about 65%, about 70%, about 75%, about 80%, about 85%, or about 90%. In some embodiments, compacting the mixture results in a relative compacted volume of the coated or enlarged aggregate particles compared to the volume prior to compaction of at most about 50%, about 55%, about 60%, about 65%, about 70%, about 75%, about 80%, about 85%, about 90%, or about 95%.

In some embodiments, compaction of the enlarged or coated aggregate particles produces a maximum average gap between adjacent particles after compaction of about about 5 μm to about 60 μm. In some embodiments, compaction of the enlarged or coated aggregate particles produces a maximum average gap between adjacent particles after compaction of about about 5 μm to about 10 μm, about 5 μm to about 15 μm, about 5 μm to about 20 μm, about 5 μm to about 25 μm, about 5μ m to about 30 μm, about 5μ m to about 35 μm, about 5 μm to about 40 μm, about 5 μm to about 45 μm, about 5 μm to about 50 μm, about 5 μm to about 55 μm, about 5 μm to about 60 μm, about 10 μm to about 15 μm, about 10 μm to about 20 μm, about 10 μm to about 25 μm, about 10 μm to about 30 μm, about 10 μm to about 35 μm, about 10 μm to about 40 μm, about 10 μm to about 45 μm, about 10 μm to about 50 μm, about 10 μm to about 55 μm, about 10 μm to about 60 μm, about 15 μm to about 20 μm, about 15 μm to about 25 μm, about 15 μm to about 30 μm, about 15 μm to about 35 μm, about 15 μm to about 40 μm, about 15 μm to about 45 μm, about 15 μm to about 50 μm, about 15 μm to about 55 μm, about 15 μm to about 60 μm, about 20 μm to about 25 μm, about 20 μm to about 30 μm, about 20 μm to about 35 μm, about 20 μm to about 40 μm, about 20 μm to about 45 μm, about 20 μm to about 50 μm, about 20 μm to about 55 μm, about 20 μm to about 60 μm, about 25 μm to about 30 μm, about 25 μm to about 35 μm, about 25 μm to about 40 μm, about 25μ m to about 45 μm, about 25μ m to about 50 μm, about 25μ m to about 55 μm, about 25 μm to about 60 μm, about 30 μm to about 35 μm, about 30 μm to about 40 μm, about 30 μm to about 45 μm, about 30 μm to about 50 μm, about 30 μm to about 55 μm, about 30 μm to about 60 μm, about 35 μm to about 40 μm, about 35 μm to about 45 μm, about 35 μm to about 50 μm, about 35 μm to about 55 μm, about 35 μm to about 60 μm, about 40 μm to about 45 μm, about 40 μm to about 50 μm, about 40 μm to about 55 μm, about 40 μm to about 60 μm, about 45 μm to about 50 μm, about 45 μm to about 55 μm, about 45 μm to about 60 μm, about 50 μm to about 55 μm, about 50 μm to about 60 μm, or about 55 μm to about 60 μm. In some embodiments, compaction of the enlarged or coated aggregate particles produces a maximum average gap between adjacent particles after compaction of about about 5 μm, about 10 μm, about 15 μm, about 20 μm, about 25 μm, about 30 μm, about 35 μm, about 40 μm, about 45 μm, about 50 μm, about 55 μm, or about 60 μm. In some embodiments, compaction of the enlarged or coated aggregate particles produces a maximum average gap between adjacent particles after compaction of about at least about 5 μm, about 10 μm, about 15 μm, about 20 μm, about 25 μm, about 30 μm, about 35 μm, about 40 μm, about 45 μm, about 50 μm, or about 55 μm. In some embodiments, compaction of the enlarged or coated aggregate particles produces a maximum average gap between adjacent particles after compaction of about at most about 10 μm, about 15 μm, about 20 μm, about 25 μm, about 30 μm, about 35 μm, about 40 μm, about 45 μm, about 50 μm, about 55 μm, or about 60 μm.

In some embodiments, the vibratory press motor is operated at rotational speed of about 60 RPM to about 7,200 RPM. In some embodiments, the vibratory press motor is operated at rotational speed of about 60 RPM to about 300 RPM, about 60 RPM to about 600 RPM, about 60 RPM to about 1,200 RPM, about 60 RPM to about 1,800 RPM, about 60 RPM to about 2,700 RPM, about 60 RPM to about 2,840 RPM, about 60 RPM to about 3,600 RPM, about 60 RPM to about 4,500 RPM, about 60 RPM to about 5,400 RPM, about 60 RPM to about 6,300 RPM, about 60 RPM to about 7,200 RPM, about 300 RPM to about 600 RPM, about 300 RPM to about 1,200 RPM, about 300 RPM to about 1,800 RPM, about 300 RPM to about 2,700 RPM, about 300 RPM to about 2,840 RPM, about 300 RPM to about 3,600 RPM, about 300 RPM to about 4,500 RPM, about 300 RPM to about 5,400 RPM, about 300 RPM to about 6,300 RPM, about 300 RPM to about 7,200 RPM, about 600 RPM to about 1,200 RPM, about 600 RPM to about 1,800 RPM, about 600 RPM to about 2,700 RPM, about 600 RPM to about 2,840 RPM, about 600 RPM to about 3,600 RPM, about 600 RPM to about 4,500 RPM, about 600 RPM to about 5,400 RPM, about 600 RPM to about 6,300 RPM, about 600 RPM to about 7,200 RPM, about 1,200 RPM to about 1,800 RPM, about 1,200 RPM to about 2,700 RPM, about 1,200 RPM to about 2,840 RPM, about 1,200 RPM to about 3,600 RPM, about 1,200 RPM to about 4,500 RPM, about 1,200 RPM to about 5,400 RPM, about 1,200 RPM to about 6,300 RPM, about 1,200 RPM to about 7,200 RPM, about 1,800 RPM to about 2,700 RPM, about 1,800 RPM to about 2,840 RPM, about 1,800 RPM to about 3,600 RPM, about 1,800 RPM to about 4,500 RPM, about 1,800 RPM to about 5,400 RPM, about 1,800 RPM to about 6,300 RPM, about 1,800 RPM to about 7,200 RPM, about 2,700 RPM to about 2,840 RPM, about 2,700 RPM to about 3,600 RPM, about 2,700 RPM to about 4,500 RPM, about 2,700 RPM to about 5,400 RPM, about 2,700 RPM to about 6,300 RPM, about 2,700 RPM to about 7,200 RPM, about 2,840 RPM to about 3,600 RPM, about 2,840 RPM to about 4,500 RPM, about 2,840 RPM to about 5,400 RPM, about 2,840 RPM to about 6,300 RPM, about 2,840 RPM to about 7,200 RPM, about 3,600 RPM to about 4,500 RPM, about 3,600 RPM to about 5,400 RPM, about 3,600 RPM to about 6,300 RPM, about 3,600 RPM to about 7,200 RPM, about 4,500 RPM to about 5,400 RPM, about 4,500 RPM to about 6,300 RPM, about 4,500 RPM to about 7,200 RPM, about 5,400 RPM to about 6,300 RPM, about 5,400 RPM to about 7,200 RPM, or about 6,300 RPM to about 7,200 RPM. In some embodiments, the vibratory press motor is operated at rotational speed of about 60 RPM, about 300 RPM, about 600 RPM, about 1,200 RPM, about 1,800 RPM, about 2,700 RPM, about 2,840 RPM, about 3,600 RPM, about 4,500 RPM, about 5,400 RPM, about 6,300 RPM, or about 7,200 RPM. In some embodiments, the vibratory press motor is operated at rotational speed of at least about 60 RPM, about 300 RPM, about 600 RPM, about 1,200 RPM, about 1,800 RPM, about 2,700 RPM, about 2,840 RPM, about 3,600 RPM, about 4,500 RPM, about 5,400 RPM, or about 6,300 RPM. In some embodiments, the vibratory press motor is operated at rotational speed of at most about 300 RPM, about 600 RPM, about 1,200 RPM, about 1,800 RPM, about 2,700 RPM, about 2,840 RPM, about 3,600 RPM, about 4,500 RPM, about 5,400 RPM, about 6,300 RPM, or about 7,200 RPM.

In some embodiments, the vibratory press motor is operated at a duty cycle of about 0.01% to about 100%. In some embodiments, the vibratory press motor is operated at a duty cycle of about 0.01% to about 0.05%, about 0.01% to about 0.1%, about 0.01% to about 1%, about 0.01% to about 2%, about 0.01% to about 3%, about 0.01% to about 5%, about 0.01% to about 10%, about 0.01% to about 30%, about 0.01% to about 50%, about 0.01% to about 70%, about 0.01% to about 100%, about 0.05% to about 0.1%, about 0.05% to about 1%, about 0.05% to about 2%, about 0.05% to about 3%, about 0.05% to about 5%, about 0.05% to about 10%, about 0.05% to about 30%, about 0.05% to about 50%, about 0.05% to about 70%, about 0.05% to about 100%, about 0.1% to about 1%, about 0.1% to about 2%, about 0.1% to about 3%, about 0.1% to about 5%, about 0.1% to about 10%, about 0.1% to about 30%, about 0.1% to about 50%, about 0.1% to about 70%, about 0.1% to about 100%, about 1% to about 2%, about 1% to about 3%, about 1% to about 5%, about 1% to about 10%, about 1% to about 30%, about 1% to about 50%, about 1% to about 70%, about 1% to about 100%, about 2% to about 3%, about 2% to about 5%, about 2% to about 10%, about 2% to about 30%, about 2% to about 50%, about 2% to about 70%, about 2% to about 100%, about 3% to about 5%, about 3% to about 10%, about 3% to about 30%, about 3% to about 50%, about 3% to about 70%, about 3% to about 100%, about 5% to about 10%, about 5% to about 30%, about 5% to about 50%, about 5% to about 70%, about 5% to about 100%, about 10% to about 30%, about 10% to about 50%, about 10% to about 70%, about 10% to about 100%, about 30% to about 50%, about 30% to about 70%, about 30% to about 100%, about 50% to about 70%, about 50% to about 100%, or about 70% to about 100%. In some embodiments, the vibratory press motor is operated at a duty cycle of about 0.01%, about 0.05%, about 0.1%, about 1%, about 2%, about 3%, about 5%, about 10%, about 30%, about 50%, about 70%, or about 100%. In some embodiments, the vibratory press motor is operated at a duty cycle of at least about 0.01%, about 0.05%, about 0.1%, about 1%, about 2%, about 3%, about 5%, about 10%, about 30%, about 50%, or about 70%. In some embodiments, the vibratory press motor is operated at a duty cycle of at most about 0.05%, about 0.1%, about 1%, about 2%, about 3%, about 5%, about 10%, about 30%, about 50%, about 70%, or about 100%.

In some embodiments, the mixer used in either the initial or the secondary cementation steps is operated at a mixing speed of about 0.5 RPM to about 200 RPM. In some embodiments, the mixer used in either the initial or the secondary cementation steps is operated at a mixing speed of about 0.5 RPM to about 1 RPM, about 0.5 RPM to about 5 RPM, about 0.5 RPM to about 10 RPM, about 0.5 RPM to about 20 RPM, about 0.5 RPM to about 30 RPM, about 0.5 RPM to about 40 RPM, about 0.5 RPM to about 50 RPM, about 0.5 RPM to about 75 RPM, about 0.5 RPM to about 100 RPM, about 0.5 RPM to about 150 RPM, about 0.5 RPM to about 200 RPM, about 1 RPM to about 5 RPM, about 1 RPM to about 10 RPM, about 1 RPM to about 20 RPM, about 1 RPM to about 30 RPM, about 1 RPM to about 40 RPM, about 1 RPM to about 50 RPM, about 1 RPM to about 75 RPM, about 1 RPM to about 100 RPM, about 1 RPM to about 150 RPM, about 1 RPM to about 200 RPM, about 5 RPM to about 10 RPM, about 5 RPM to about 20 RPM, about 5 RPM to about 30 RPM, about 5 RPM to about 40 RPM, about 5 RPM to about 50 RPM, about 5 RPM to about 75 RPM, about 5 RPM to about 100 RPM, about 5 RPM to about 150 RPM, about 5 RPM to about 200 RPM, about 10 RPM to about 20 RPM, about 10 RPM to about 30 RPM, about 10 RPM to about 40 RPM, about 10 RPM to about 50 RPM, about 10 RPM to about 75 RPM, about 10 RPM to about 100 RPM, about 10 RPM to about 150 RPM, about 10 RPM to about 200 RPM, about 20 RPM to about 30 RPM, about 20 RPM to about 40 RPM, about 20 RPM to about 50 RPM, about 20 RPM to about 75 RPM, about 20 RPM to about 100 RPM, about 20 RPM to about 150 RPM, about 20 RPM to about 200 RPM, about 30 RPM to about 40 RPM, about 30 RPM to about 50 RPM, about 30 RPM to about 75 RPM, about 30 RPM to about 100 RPM, about 30 RPM to about 150 RPM, about 30 RPM to about 200 RPM, about 40 RPM to about 50 RPM, about 40 RPM to about 75 RPM, about 40 RPM to about 100 RPM, about 40 RPM to about 150 RPM, about 40 RPM to about 200 RPM, about 50 RPM to about 75 RPM, about 50 RPM to about 100 RPM, about 50 RPM to about 150 RPM, about 50 RPM to about 200 RPM, about 75 RPM to about 100 RPM, about 75 RPM to about 150 RPM, about 75 RPM to about 200 RPM, about 100 RPM to about 150 RPM, about 100 RPM to about 200 RPM, or about 150 RPM to about 200 RPM. In some embodiments, the mixer used in either the initial or the secondary cementation steps is operated at a mixing speed of about 0.5 RPM, about 1 RPM, about 5 RPM, about 10 RPM, about 20 RPM, about 30 RPM, about 40 RPM, about 50 RPM, about 75 RPM, about 100 RPM, about 150 RPM, or about 200 RPM. In some embodiments, the mixer used in either the initial or the secondary cementation steps is operated at a mixing speed of at least about 0.5 RPM, about 1 RPM, about 5 RPM, about 10 RPM, about 20 RPM, about 30 RPM, about 40 RPM, about 50 RPM, about 75 RPM, about 100 RPM, or about 150 RPM. In some embodiments, the mixer used in either the initial or the secondary cementation steps is operated at a mixing speed of at most about 1 RPM, about 5 RPM, about 10 RPM, about 20 RPM, about 30 RPM, about 40 RPM, about 50 RPM, about 75 RPM, about 100 RPM, about 150 RPM, or about 200 RPM.

In some embodiments, the mixer used in either the initial or the secondary cementation steps is operated at a duty cycle of about 0.01% to about 100%. In some embodiments, the mixer used in either the initial or the secondary cementation steps is operated at a duty cycle of about 0.01% to about 0.05%, about 0.01% to about 0.1%, about 0.01% to about 1%, about 0.01% to about 2%, about 0.01% to about 3%, about 0.01% to about 5%, about 0.01% to about 10%, about 0.01% to about 30%, about 0.01% to about 50%, about 0.01% to about 70%, about 0.01% to about 100%, about 0.05% to about 0.1%, about 0.05% to about 1%, about 0.05% to about 2%, about 0.05% to about 3%, about 0.05% to about 5%, about 0.05% to about 10%, about 0.05% to about 30%, about 0.05% to about 50%, about 0.05% to about 70%, about 0.05% to about 100%, about 0.1% to about 1%, about 0.1% to about 2%, about 0.1% to about 3%, about 0.1% to about 5%, about 0.1% to about 10%, about 0.1% to about 30%, about 0.1% to about 50%, about 0.1% to about 70%, about 0.1% to about 100%, about 1% to about 2%, about 1% to about 3%, about 1% to about 5%, about 1% to about 10%, about 1% to about 30%, about 1% to about 50%, about 1% to about 70%, about 1% to about 100%, about 2% to about 3%, about 2% to about 5%, about 2% to about 10%, about 2% to about 30%, about 2% to about 50%, about 2% to about 70%, about 2% to about 100%, about 3% to about 5%, about 3% to about 10%, about 3% to about 30%, about 3% to about 50%, about 3% to about 70%, about 3% to about 100%, about 5% to about 10%, about 5% to about 30%, about 5% to about 50%, about 5% to about 70%, about 5% to about 100%, about 10% to about 30%, about 10% to about 50%, about 10% to about 70%, about 10% to about 100%, about 30% to about 50%, about 30% to about 70%, about 30% to about 100%, about 50% to about 70%, about 50% to about 100%, or about 70% to about 100%. In some embodiments, the mixer used in either the initial or the secondary cementation steps is operated at a duty cycle of about 0.01%, about 0.05%, about 0.1%, about 1%, about 2%, about 3%, about 5%, about 10%, about 30%, about 50%, about 70%, or about 100%. In some embodiments, the mixer used in either the initial or the secondary cementation steps is operated at a duty cycle of at least about 0.01%, about 0.05%, about 0.1%, about 1%, about 2%, about 3%, about 5%, about 10%, about 30%, about 50%, or about 70%. In some embodiments, the mixer used in either the initial or the secondary cementation steps is operated at a duty cycle of at most about 0.05%, about 0.1%, about 1%, about 2%, about 3%, about 5%, about 10%, about 30%, about 50%, about 70%, or about 100%.

In some embodiments, the vibratory press applies a compressive force of about 10 psi to about 40,000 psi. In some embodiments, the vibratory press applies a compressive force of about 10 psi to about 50 psi, about 10 psi to about 100 psi, about 10 psi to about 300 psi, about 10 psi to about 600 psi, about 10 psi to about 1,200 psi, about 10 psi to about 2,400 psi, about 10 psi to about 4,800 psi, about 10 psi to about 7,500 psi, about 10 psi to about 10,000 psi, about 10 psi to about 20,000 psi, about 10 psi to about 40,000 psi, about 50 psi to about 100 psi, about 50 psi to about 300 psi, about 50 psi to about 600 psi, about 50 psi to about 1,200 psi, about 50 psi to about 2,400 psi, about 50 psi to about 4,800 psi, about 50 psi to about 7,500 psi, about 50 psi to about 10,000 psi, about 50 psi to about 20,000 psi, about 50 psi to about 40,000 psi, about 100 psi to about 300 psi, about 100 psi to about 600 psi, about 100 psi to about 1,200 psi, about 100 psi to about 2,400 psi, about 100 psi to about 4,800 psi, about 100 psi to about 7,500 psi, about 100 psi to about 10,000 psi, about 100 psi to about 20,000 psi, about 100 psi to about 40,000 psi, about 300 psi to about 600 psi, about 300 psi to about 1,200 psi, about 300 psi to about 2,400 psi, about 300 psi to about 4,800 psi, about 300 psi to about 7,500 psi, about 300 psi to about 10,000 psi, about 300 psi to about 20,000 psi, about 300 psi to about 40,000 psi, about 600 psi to about 1,200 psi, about 600 psi to about 2,400 psi, about 600 psi to about 4,800 psi, about 600 psi to about 7,500 psi, about 600 psi to about 10,000 psi, about 600 psi to about 20,000 psi, about 600 psi to about 40,000 psi, about 1,200 psi to about 2,400 psi, about 1,200 psi to about 4,800 psi, about 1,200 psi to about 7,500 psi, about 1,200 psi to about 10,000 psi, about 1,200 psi to about 20,000 psi, about 1,200 psi to about 40,000 psi, about 2,400 psi to about 4,800 psi, about 2,400 psi to about 7,500 psi, about 2,400 psi to about 10,000 psi, about 2,400 psi to about 20,000 psi, about 2,400 psi to about 40,000 psi, about 4,800 psi to about 7,500 psi, about 4,800 psi to about 10,000 psi, about 4,800 psi to about 20,000 psi, about 4,800 psi to about 40,000 psi, about 7,500 psi to about 10,000 psi, about 7,500 psi to about 20,000 psi, about 7,500 psi to about 40,000 psi, about 10,000 psi to about 20,000 psi, about 10,000 psi to about 40,000 psi, or about 20,000 psi to about 40,000 psi. In some embodiments, the vibratory press applies a compressive force of about 10 psi, about 50 psi, about 100 psi, about 300 psi, about 600 psi, about 1,200 psi, about 2,400 psi, about 4,800 psi, about 7,500 psi, about 10,000 psi, about 20,000 psi, or about 40,000 psi. In some embodiments, the vibratory press applies a compressive force of at least about 10 psi, about 50 psi, about 100 psi, about 300 psi, about 600 psi, about 1,200 psi, about 2,400 psi, about 4,800 psi, about 7,500 psi, about 10,000 psi, or about 20,000 psi. In some embodiments, the vibratory press applies a compressive force of at most about 50 psi, about 100 psi, about 300 psi, about 600 psi, about 1,200 psi, about 2,400 psi, about 4,800 psi, about 7,500 psi, about 10,000 psi, about 20,000 psi, or about 40,000 psi.

In some embodiments, the average particle size of the aggregate particles is increased by the initial cementation reaction by about 10% to about 1,000%. In some embodiments, the average particle size of the aggregate particles is increased by the initial cementation reaction by about 10% to about 20%, about 10% to about 30%, about 10% to about 50%, about 10% to about 75%, about 10% to about 100%, about 10% to about 150%, about 10% to about 200%, about 10% to about 300%, about 10% to about 500%, about 10% to about 700%, about 10% to about 1,000%, about 20% to about 30%, about 20% to about 50%, about 20% to about 75%, about 20% to about 100%, about 20% to about 150%, about 20% to about 200%, about 20% to about 300%, about 20% to about 500%, about 20% to about 700%, about 20% to about 1,000%, about 30% to about 50%, about 30% to about 75%, about 30% to about 100%, about 30% to about 150%, about 30% to about 200%, about 30% to about 300%, about 30% to about 500%, about 30% to about 700%, about 30% to about 1,000%, about 50% to about 75%, about 50% to about 100%, about 50% to about 150%, about 50% to about 200%, about 50% to about 300%, about 50% to about 500%, about 50% to about 700%, about 50% to about 1,000%, about 75% to about 100%, about 75% to about 150%, about 75% to about 200%, about 75% to about 300%, about 75% to about 500%, about 75% to about 700%, about 75% to about 1,000%, about 100% to about 150%, about 100% to about 200%, about 100% to about 300%, about 100% to about 500%, about 100% to about 700%, about 100% to about 1,000%, about 150% to about 200%, about 150% to about 300%, about 150% to about 500%, about 150% to about 700%, about 150% to about 1,000%, about 200% to about 300%, about 200% to about 500%, about 200% to about 700%, about 200% to about 1,000%, about 300% to about 500%, about 300% to about 700%, about 300% to about 1,000%, about 500% to about 700%, about 500% to about 1,000%, or about 700% to about 1,000%. In some embodiments, the average particle size of the aggregate particles is increased by the initial cementation reaction by about 10%, about 20%, about 30%, about 50%, about 75%, about 100%, about 150%, about 200%, about 300%, about 500%, about 700%, or about 1,000%. In some embodiments, the average particle size of the aggregate particles is increased by the initial cementation reaction by at least about 10%, about 20%, about 30%, about 50%, about 75%, about 100%, about 150%, about 200%, about 300%, about 500%, or about 700%. In some embodiments, the average particle size of the aggregate particles is increased by the initial cementation reaction by at most about 20%, about 30%, about 50%, about 75%, about 100%, about 150%, about 200%, about 300%, about 500%, about 700%, or about 1,000%.

In some embodiments, the aggregate particles are each individually increased in size by the initial cementation reaction radially by about 10 μm to about 200 μm. In some embodiments, the aggregate particles are each individually increased in size by the initial cementation reaction radially by about 10 μm to about 20 μm, about 10 μm to about 50 μm, about 10 μm to about 60 μm, about 10 μm to about 70 μm, about 10 μm to about 80 μm, about 10 μm to about 90 μm, about 10 μm to about 100 μm, about 10 μm to about 125 μm, about 10 μm to about 150 μm, about 10 μm to about 175 μm, about 10 μm to about 200 μm, about 20 μm to about 50 μm, about 20 μm to about 60 μm, about 20 μm to about 70 μm, about 20 μm to about 80 μm, about 20 μm to about 90 μm, about 20 μm to about 100 μm, about 20 μm to about 125 μm, about 20 μm to about 150 μm, about 20 μm to about 175 μm, about 20 μm to about 200 μm, about 50 μm to about 60 μm, about 50 μm to about 70 μm, about 50 μm to about 80 μm, about 50 μm to about 90 μm, about 50 μm to about 100 μm, about 50 μm to about 125 μm, about 50 μm to about 150 μm, about 50 μm to about 175 μm, about 50 μm to about 200 μm, about 60 μm to about 70 μm, about 60 μm to about 80 μm, about 60 μm to about 90 μm, about 60 μm to about 100 μm, about 60 μm to about 125 μm, about 60 μm to about 150 μm, about 60 μm to about 175 μm, about 60 μm to about 200 μm, about 70 μm to about 80 μm, about 70 μm to about 90 μm, about 70 μm to about 100 μm, about 70 μm to about 125 μm, about 70 μm to about 150 μm, about 70μ m to about 175 μm, about 70μ m to about 200 μm, about 80μ m to about 90 μm, about 80 μm to about 100 μm, about 80 μm to about 125 μm, about 80 μm to about 150 μm, about 80 μm to about 175 μm, about 80 μm to about 200 μm, about 90 μm to about 100 μm, about 90 μm to about 125 μm, about 90 μm to about 150 μm, about 90 μm to about 175 μm, about 90 μm to about 200 μm, about 100 μm to about 125 μm, about 100 μm to about 150 μm, about 100 μm to about 175 μm, about 100 μm to about 200 μm, about 125 μm to about 150 μm, about 125 μm to about 175 μm, about 125 μm to about 200 μm, about 150 μm to about 175 μm, about 150 μm to about 200 μm, or about 175 μm to about 200 μm. In some embodiments, the aggregate particles are each individually increased in size by the initial cementation reaction radially by about 10 μm, about 20 μm, about 50 μm, about 60 μm, about 70 μm, about 80 μm, about 90 μm, about 100 μm, about 125 μm, about 150 μm, about 175 μm, or about 200 μm. In some embodiments, the aggregate particles are each individually increased in size by the initial cementation reaction radially by at least about 10 μm, about 20 μm, about 50 μm, about 60 μm, about 70 μm, about 80 μm, about 90 μm, about 100 μm, about 125 μm, about 150 μm, or about 175 μm. In some embodiments, the aggregate particles are increased in size radially by at most about 20 μm, about 50 μm, about 60 μm, about 70 μm, about 80 μm, about 90 μm, about 100 μm, about 125 μm, about 150 μm, about 175 μm, or about 200 μm.

In some embodiments, the concentration of cells of the biological organism in the solution added to the aggregate to perform either the initial or secondary cementation reaction is about 10,000 CFU/mL to about 1,000,000,000,000 CFU/mL. In some embodiments, the concentration of cells of the biological organism in the solution added to the aggregate to perform either the initial or secondary cementation reaction is about 10,000 CFU/mL to about 100,000 CFU/mL, about 10,000 CFU/mL to about 1,000,000 CFU/mL, about 10,000 CFU/mL to about 10,000,000 CFU/mL, about 10,000 CFU/mL to about 100,000,000 CFU/mL, about 10,000 CFU/mL to about 1,000,000,000 CFU/mL, about 10,000 CFU/mL to about 10,000,000,000 CFU/mL, about 10,000 CFU/mL to about 100,000,000,000 CFU/mL, about 10,000 CFU/mL to about 1,000,000,000,000 CFU/mL, about 100,000 CFU/mL to about 1,000,000 CFU/mL, about 100,000 CFU/mL to about 10,000,000 CFU/mL, about 100,000 CFU/mL to about 100,000,000 CFU/mL, about 100,000 CFU/mL to about 1,000,000,000 CFU/mL, about 100,000 CFU/mL to about 10,000,000,000 CFU/mL, about 100,000 CFU/mL to about 100,000,000,000 CFU/mL, about 100,000 CFU/mL to about 1,000,000,000,000 CFU/mL, about 1,000,000 CFU/mL to about 10,000,000 CFU/mL, about 1,000,000 CFU/mL to about 100,000,000 CFU/mL, about 1,000,000 CFU/mL to about 1,000,000,000 CFU/mL, about 1,000,000 CFU/mL to about 10,000,000,000 CFU/mL, about 1,000,000 CFU/mL to about 100,000,000,000 CFU/mL, about 1,000,000 CFU/mL to about 1,000,000,000,000 CFU/mL, about 10,000,000 CFU/mL to about 100,000,000 CFU/mL, about 10,000,000 CFU/mL to about 1,000,000,000 CFU/mL, about 10,000,000 CFU/mL to about 10,000,000,000 CFU/mL, about 10,000,000 CFU/mL to about 100,000,000,000 CFU/mL, about 10,000,000 CFU/mL to about 1,000,000,000,000 CFU/mL, about 100,000,000 CFU/mL to about 1,000,000,000 CFU/mL, about 100,000,000 CFU/mL to about 10,000,000,000 CFU/mL, about 100,000,000 CFU/mL to about 100,000,000,000 CFU/mL, about 100,000,000 CFU/mL to about 1,000,000,000,000 CFU/mL, about 1,000,000,000 CFU/mL to about 10,000,000,000 CFU/mL, about 1,000,000,000 CFU/mL to about 100,000,000,000 CFU/mL, about 1,000,000,000 CFU/mL to about 1,000,000,000,000 CFU/mL, about 10,000,000,000 CFU/mL to about 100,000,000,000 CFU/mL, about 10,000,000,000 CFU/mL to about 1,000,000,000,000 CFU/mL, or about 100,000,000,000 CFU/mL to about 1,000,000,000,000 CFU/mL. In some embodiments, the concentration of cells of the biological organism in the solution added to the aggregate to perform either the initial or secondary cementation reaction is about 10,000 CFU/mL, about 100,000 CFU/mL, about 1,000,000 CFU/mL, about 10,000,000 CFU/mL, about 100,000,000 CFU/mL, about 1,000,000,000 CFU/mL, about 10,000,000,000 CFU/mL, about 100,000,000,000 CFU/mL, or about 1,000,000,000,000 CFU/mL. In some embodiments, the concentration of cells of the biological organism in the solution added to the aggregate to perform either the initial or secondary cementation reaction is at least about 10,000 CFU/mL, about 100,000 CFU/mL, about 1,000,000 CFU/mL, about 10,000,000 CFU/mL, about 100,000,000 CFU/mL, about 1,000,000,000 CFU/mL, about 10,000,000,000 CFU/mL, or about 100,000,000,000 CFU/mL. In some embodiments, the concentration of cells of the biological organism in the solution added to the aggregate to perform either the initial or secondary cementation reaction is at most about 100,000 CFU/mL, about 1,000,000 CFU/mL, about 10,000,000 CFU/mL, about 100,000,000 CFU/mL, about 1,000,000,000 CFU/mL, about 10,000,000,000 CFU/mL, about 100,000,000,000 CFU/mL, or about 1,000,000,000,000 CFU/mL.

### Biocement Mixing Devices

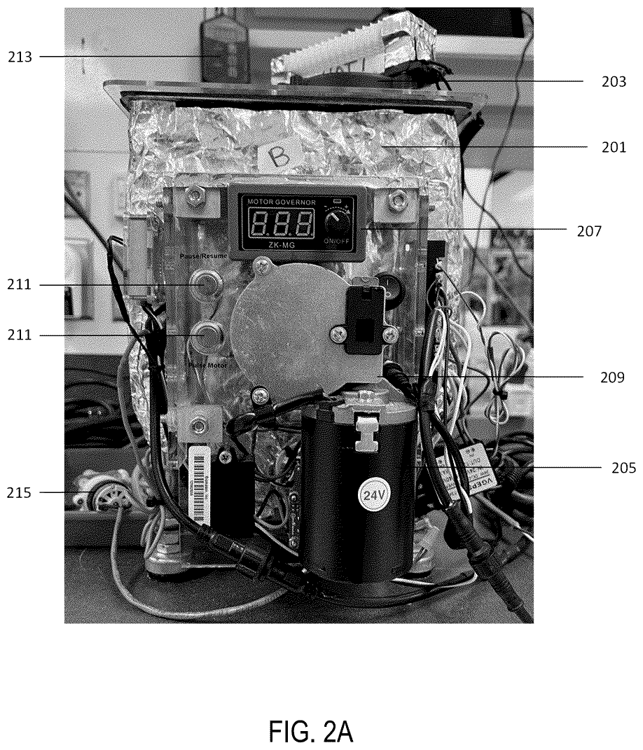

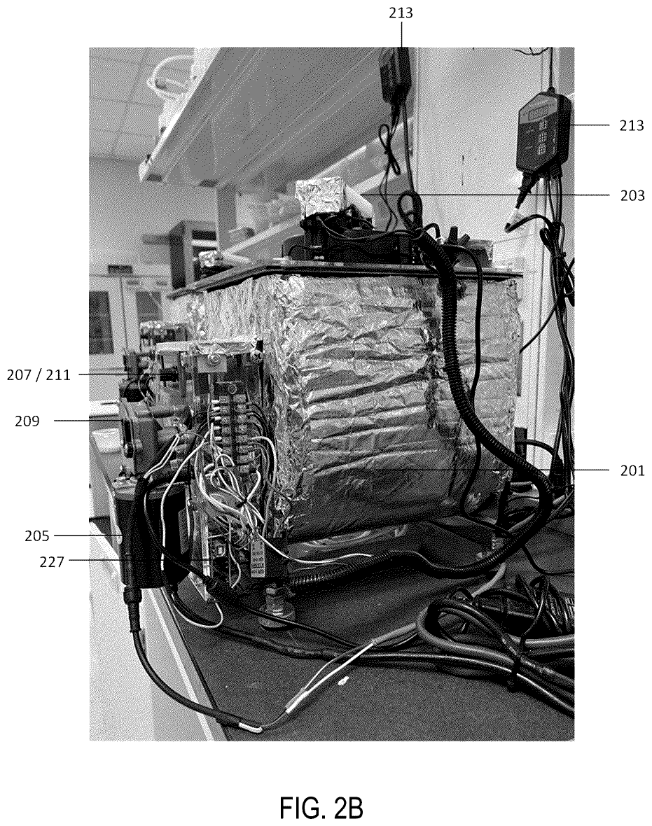

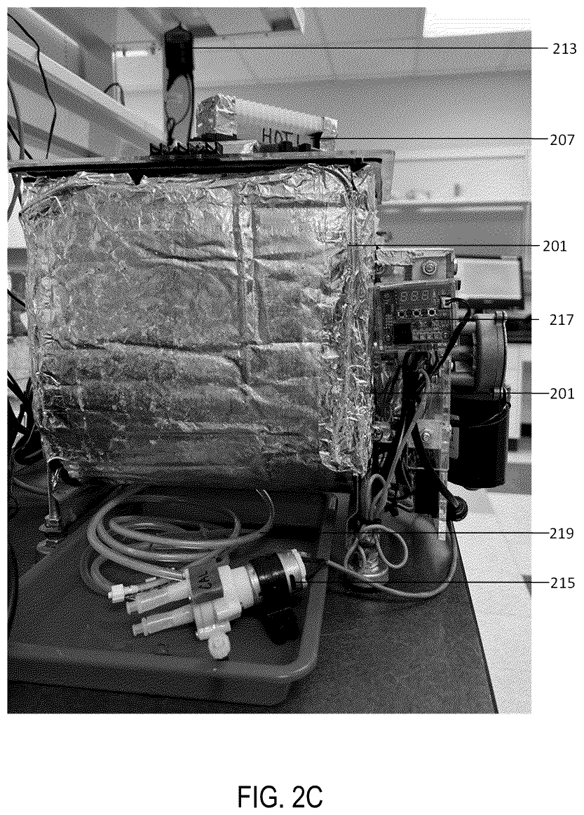

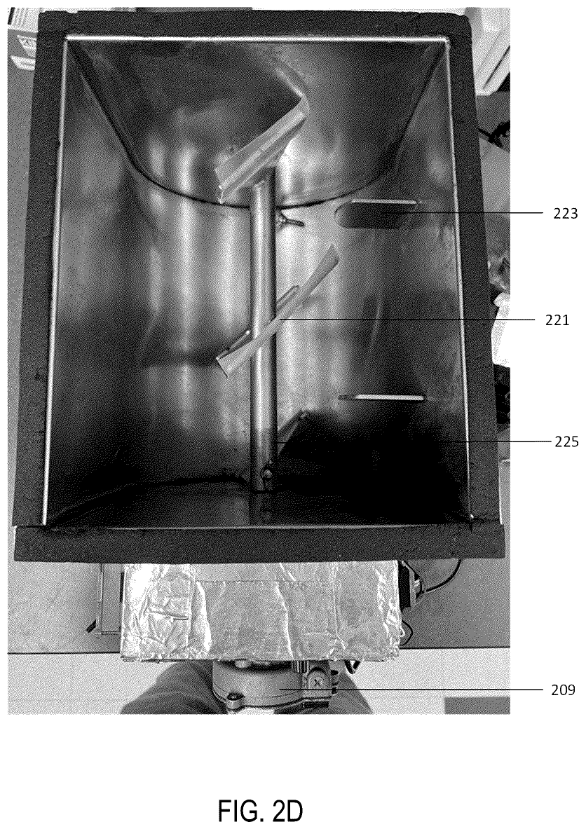

In another aspect described herein, are specialized mixing devices for producing bioconcrete construction materials without the need for hydroponics. An example mixing device is depicted in FIGS. 2A-D. In some embodiments, the mixing device comprises a mixing tank 201. The mixing tank may be thermally insulated or may comprise one or more heating elements. In some embodiments, the mixing device comprises a plurality of mixing tines 221 and 223. The plurality of mixing tines may comprise movable tines 221, fixed tines 223, or both. In some embodiments, the mixing device comprises one or more mixing motors 205. The mixing motors may be operably coupled to the mixing tines either directly by a driveshaft 225, through a gearbox 209, or other mechanical means, and to one or more motor controllers 207. The motor controllers may be programmable to set a duty cycle and motor speed of the mixer.

In some embodiments, the mixing device comprises a reagent delivery pump 215. In some embodiments, the reagent delivery pump is operably coupled to the mixing tank by a reagent line 219. In some embodiments, the delivery line is made from flexible pump tubing. In some embodiments, the reagent delivery pump is configured infuse a measured quantity of a reagent from a reservoir 213 into the mixing tank. In some embodiments, the reagent delivery pump is configured to deliver reagents in liquid solutions. In some embodiments, the reagent delivery pump is configured to deliver solid reagents. The mixing device may comprise a pump controller, operably coupled to the reagent delivery pump, which may be programmable to set timing and/or dose amounts of reagent delivery.

In some embodiments, the mixing device comprises a heater 203 and a temperature sensor configured to measure the temperature inside the mixing tank. The temperature sensor and heater may be operably coupled to a temperature controller 213, which is programmable to maintain a set temperature or to create a temperature gradient over time within the mixing tank. In some embodiments, the mixing device comprises a main controller 227, which communicates with the other controllers to coordinate inputs and outputs of each of the subsystems. In some embodiments, each of the controllers may independently be coupled to one or more optional user input devices 211, which allow a user to adjust controller settings.

In some embodiments, the mixing device comprises a humidity sensor and humidity controller, which are programmable to monitor and/or maintain the humidity inside of the mixing tank. In some embodiments, the mixing device comprises a pH sensor or ion selective electrode and a pH controller, which are programmable to monitor and/or maintain or adjust the pH or another reaction parameter within the mixing tank while the device is in use. In some embodiments, the mixing tank comprises a drain, which allows excess liquid to be removed during operation. In some embodiments, the mixing device comprises additional reagent pumps for use in complex control schemes. In some embodiments, the mixing device comprises additional heaters and/or temperature sensors for maintaining or controlling the temperature of the one or more reagent reservoirs, the one or more reagent lines, or one or more water input lines. In some embodiments, the mixing device comprises one or more valves for adjusting the moisture level inside the mixing tank. The one or more valves may comprise a drain valve and or a water line valve.

In some embodiments, the mixing tank has a capacity of about 0.01 yd³ to about 30 yd³. In some embodiments, the mixing tank has a capacity of about 0.01 yd³ to about 0.1 yd³, about 0.01 yd³ to about 0.5 yd³, about 0.01 yd³ to about 0.75 yd³, about 0.01 yd³ to about 1 yd³, about 0.01 yd³ to about 2 yd³, about 0.01 yd³ to about 3 yd³, about 0.01 yd³ to about 4 yd³, about 0.01 yd³ to about 8 yd³, about 0.01 yd³ to about 10 yd³, about 0.01 yd³ to about 20 yd³, about 0.01 yd³ to about 30 yd³, about 0.1 yd³ to about 0.5 yd³, about 0.1 yd³ to about 0.75 yd³, about 0.1 yd³ to about 1 yd³, about 0.1 yd³ to about 2 yd³, about 0.1 yd³ to about 3 yd³, about 0.1 yd³ to about 4 yd³, about 0.1 yd³ to about 8 yd³, about 0.1 yd³ to about 10 yd³, about 0.1 yd³ to about 20 yd³, about 0.1 yd³ to about 30 yd³, about 0.5 yd³ to about 0.75 yd³, about 0.5 yd³ to about 1 yd³, about 0.5 yd³ to about 2 yd³, about 0.5 yd³ to about 3 yd³, about 0.5 yd³ to about 4 yd³, about 0.5 yd³ to about 8 yd³, about 0.5 yd³ to about 10 yd³, about 0.5 yd³ to about 20 yd³, about 0.5 yd³ to about 30 yd³, about 0.75 yd³ to about 1 yd³, about 0.75 yd³ to about 2 yd³, about 0.75 yd³ to about 3 yd³, about 0.75 yd³ to about 4 yd³, about 0.75 yd³ to about 8 yd³, about 0.75 yd³ to about 10 yd³, about 0.75 yd³ to about 20 yd³, about 0.75 yd³ to about 30 yd³, about 1 yd³ to about 2 yd³, about 1 yd³ to about 3 yd³, about 1 yd³ to about 4 yd³, about 1 yd³ to about 8 yd³, about 1 yd³ to about 10 yd³, about 1 yd³ to about 20 yd³, about 1 yd³ to about 30 yd³, about 2 yd³ to about 3 yd³, about 2 yd³ to about 4 yd³, about 2 yd³ to about 8 yd³, about 2 yd³ to about 10 yd³, about 2 yd³ to about 20 yd³, about 2 yd³ to about 30 yd³, about 3 yd³ to about 4 yd³, about 3 yd³ to about 8 yd³, about 3 yd³ to about 10 yd³, about 3 yd³ to about 20 yd³, about 3 yd³ to about 30 yd³, about 4 yd³ to about 8 yd³, about 4 yd³ to about 10 yd³, about 4 yd³ to about 20 yd³, about 4 yd³ to about 30 yd³, about 8 yd³ to about 10 yd³, about 8 yd³ to about 20 yd³, about 8 yd³ to about 30 yd³, about 10 yd³ to about 20 yd³, about 10 yd³ to about 30 yd³, or about 20 yd³ to about 30 yd³. In some embodiments, the mixing tank has a capacity of about 0.01 yd³, about 0.1 yd³, about 0.5 yd³, about 0.75 yd³, about 1 yd³, about 2 yd³, about 3 yd³, about 4 yd³, about 8 yd³, about 10 yd³, about 20 yd³, or about 30 yd³. In some embodiments, the mixing tank has a capacity of at least about 0.01 yd³, about 0.1 yd³, about 0.5 yd³, about 0.75 yd³, about 1 yd³, about 2 yd³, about 3 yd³, about 4 yd³, about 8 yd³, about 10 yd³, or about 20 yd³. In some embodiments, the mixing tank has a capacity of at most about 0.1 yd³, about 0.5 yd³, about 0.75 yd³, about 1 yd³, about 2 yd³, about 3 yd³, about 4 yd³, about 8 yd³, about 10 yd³, about 20 yd³, or about 30 yd³.

In some embodiments, heat is applied uniformly to avoid hotspots. Mixers may have jacketed heaters installed, which may use a circulated liquid heat exchange fluid such as water to deliver heat. Heat may be applied via forced air added to the headspace of the mixer.

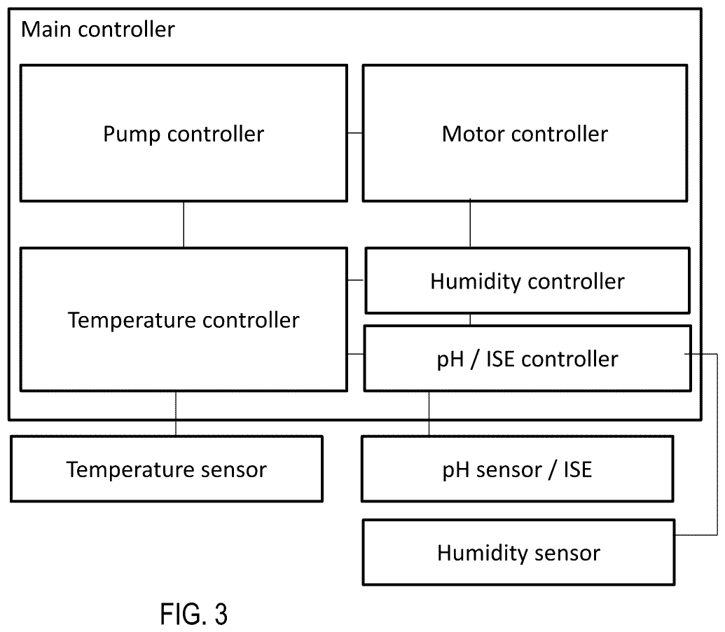

An example control scheme that may be used by a mixing device described herein is detailed in FIG. 3. The pump controller, motor controller, temperature controller, humidity controller, and pH/ion selective electrode controller may be individual components which communicate with the main controller, or may be implemented as subsystems of the main controller. Communication with between the subsystems and the sensors may be as described in the figure, e.g. each sensor directly communicating with its respective control module, or may be communicate with the main controller, which then relays data to the respective control module or subsystem.

In some embodiments, the temperature controller may utilize a closed-loop PID algorithm. In some embodiments, the control algorithms of each control module may be user programmable.

In some embodiments of a mixing device described herein, mixer surfaces in contact with process materials are compatible with high concentrations of calcium chloride, urea, and ammonia typically exhibited by biocementation processes. Examples of compatible materials are stainless steel, polymers, ceramics, resins, and/or special coatings applied to non-compatible materials.

### Biocement Compositions Comprising Supplemental Materials

Described herein are bioconcrete construction materials comprising aggregate particles, a biocement, and a supplemental material. The biocement may comprise calcium carbonate or metal carbonates useful in binding aggregate to form a bioconcrete. The biocement may be formed by a biological mechanism. The biological mechanism can comprise microbially induced carbonate precipitation relying on one or more microorganisms. The biological mechanism can comprise direct precipitation by one or more enzymes.

The aggregate material may comprise any type of natural rock or stone, glass, fiberglass, wood, biomass, paper, metal, plastic, polymers, rubber, imitation rubber, vinyl, minerals, imitations of rock or stone, recycled materials such as, for example, recycled brick, concrete, stone, mine tailings and mining residues, scrubber wastes, and/or combinations thereof. Aggregate can be of any size including mixtures of sizes provided such aggregate is smaller in size than the resulting structure. Aggregate materials may comprise particles of 10 mm or less, 5 mm or less, 1 mm or less, 0.5 mm or less, or combinations thereof. Mesh sizes can be as desired including but not limited to very fine particles (any number between and including 32-300 standard mesh), fine particles (any number between and including 10-32 standard mesh), medium particles (less than 10 standard mesh including all mesh numbers therein), and coarse particles (particles greater than or equal to 2 mm), and combinations thereof. Particle can be most any shape including, for example, round or rounded, oval, spherical (S grade), square, rectangular, hedral, fiber, lath, angular, elongated, needle-like, acicular, flat, flaky, cylindrical, spongy, cubic, cubical and combinations and variations thereof. If desired or necessary, aggregate particles can be roughened to create cracks in and crevices on particle surfaces.

The aggregate material may comprise rock (e.g., fines), sand, glass, wood, paper, metal, plastic, polymers, minerals, manufacturing or processing waste materials such as ash, carbon or wood residuals, any of which can be crushed or used whole or combinations thereof.

The aggregate may comprise organic or inorganic material such as, for example, sand, rock, glass (e.g. Poraver), wood, paper, metal, plastic, polymers, minerals, recycled materials, or combinations thereof. Aggregate particles may comprise beads, grains, rods, strands, fibers, flakes, crystals, pulverized or crushed materials, or combinations thereof.

Aggregate particles may have a diameter (e.g., actual, average or effective diameter) of about 50 mm or less, preferably about 25 mm or less, preferably about 20 mm or less, preferably about 10 mm or less, and preferably about 5 mm or less. In some embodiments, aggregate material may be about 1 mm or less and about 0.5 mm or less, about 0.1 mm or less, and about 50 μm or less. Particles sizes may include from about 10 μm to about 1 mm, from about 100 μm to about 0.5 mm, from about 200 μm to about 1 mm, from about 1 μm to about 200 μm, from about 10 nm to about 1 μm, and from about 10 nm to about 40 nm, and various combinations thereof. Aggregate particles may be largely composed of particulates of less than 5 mm in diameter (e.g. less than or about 4 mm, less than or about 3 mm, less than or about 2 mm, or less than or about 1 mm);

Aggregate particles may be characterized by fine size and may be equal to or less than 250 micron, equal to or less than 200 micron, equal to or less than 150 micron, or equal to or less than 100 micron (reference examples include micron size of beach sand=700, micron size of fine sand=250; micron size of Portland cement=74; micron size of silt=44; micron size of smoke=2).

In some embodiments large particles may be used, e.g. aggregates comprising particles of cm or mm size, e.g. gravels, stones, crushed rocks etc. In some embodiments, the size range of particles may range from 1 μm, or 10 μm, to 5 cm, or more. In some embodiments, the particulate starting material may consist of, or at least essentially consist of, small particles, in particular particles on a μm scale. In other words, the particles may be between, or in the range of 1 μm and 1 mm (1000 μm) in size, for instance between, or in the range of, 100 and 1000 μm or 100 and 500 μm, for example 200 and 400 μm, or 200 and 300 μm. Large size particles may however be, for instance, between 1 mm and 2 mm in size, or have a wider range in size, for instance between 100 μm and 2 mm.

The biocement formed in a cementation reaction may bond to individual aggregate particles to create bridges between the particles or it may encapsulate individual particles. The enzyme or enzyme-producing biological organism and the biocementation reagents may be added together or separately in the cementation reagents. The biocement may be a precipitated calcium carbonate biocement. The enzyme producing biological organism may be a urease-producing microorganism.

The urease-producing microorganism may be selected from the group of: Sporosarcina pasteurii, Sporosarcina ureae, Proteus vulgaris, Bacillus sphaericus, Myxococcus xanthus, Proteus mirabilis, Bacillus megaterium, Helicobacter pylori, and combinations of two or more thereof.

The enzyme producing biological organism may be an acid producing microorganism.

The acid-producing microorganism may be selected from the group consisting of: Variovorax, Klebsiella, Pseudomonas, Bacillus, Exiguobacterium, Microbacterium, Curtobacterium, Rathayibacter, CellFimi2, Streptomyces, Raoultella, B. pumilus, B. safanensis, B. simplex, B. licheniformis, and combinations thereof.

In some embodiments, addition of a supplemental material increases the compressive strength of a finished bioconcrete compared to a bioconcrete without a supplemental material by about 1% to about 200%. In some embodiments, addition of a supplemental material increases the compressive strength of a finished bioconcrete compared to a bioconcrete without a supplemental material by about 1% to about 2%, about 1% to about 3%, about 1% to about 5%, about 1% to about 7%, about 1% to about 10%, about 1% to about 15%, about 1% to about 20%, about 1% to about 30%, about 1% to about 50%, about 1% to about 100%, about 1% to about 200%, about 2% to about 3%, about 2% to about 5%, about 2% to about 7%, about 2% to about 10%, about 2% to about 15%, about 2% to about 20%, about 2% to about 30%, about 2% to about 50%, about 2% to about 100%, about 2% to about 200%, about 3% to about 5%, about 3% to about 7%, about 3% to about 10%, about 3% to about 15%, about 3% to about 20%, about 3% to about 30%, about 3% to about 50%, about 3% to about 100%, about 3% to about 200%, about 5% to about 7%, about 5% to about 10%, about 5% to about 15%, about 5% to about 20%, about 5% to about 30%, about 5% to about 50%, about 5% to about 100%, about 5% to about 200%, about 7% to about 10%, about 7% to about 15%, about 7% to about 20%, about 7% to about 30%, about 7% to about 50%, about 7% to about 100%, about 7% to about 200%, about 10% to about 15%, about 10% to about 20%, about 10% to about 30%, about 10% to about 50%, about 10% to about 100%, about 10% to about 200%, about 15% to about 20%, about 15% to about 30%, about 15% to about 50%, about 15% to about 100%, about 15% to about 200%, about 20% to about 30%, about 20% to about 50%, about 20% to about 100%, about 20% to about 200%, about 30% to about 50%, about 30% to about 100%, about 30% to about 200%, about 50% to about 100%, about 50% to about 200%, or about 100% to about 200%. In some embodiments, addition of a supplemental material increases the compressive strength of a finished bioconcrete compared to a bioconcrete without a supplemental material by about 1%, about 2%, about 3%, about 5%, about 7%, about 10%, about 15%, about 20%, about 30%, about 50%, about 100%, or about 200%. In some embodiments, addition of a supplemental material increases the compressive strength of a finished bioconcrete compared to a bioconcrete without a supplemental material by at least about 1%, about 2%, about 3%, about 5%, about 7%, about 10%, about 15%, about 20%, about 30%, about 50%, or about 100%. In some embodiments, addition of a supplemental material increases the compressive strength of a finished bioconcrete compared to a bioconcrete without a supplemental material by at most about 2%, about 3%, about 5%, about 7%, about 10%, about 15%, about 20%, about 30%, about 50%, about 100%, or about 200%.

In some embodiments, a bioconcrete construction material can comprise about 0.1 weight % of a supplemental material to about 40 weight % of a supplemental material. In some embodiments, a bioconcrete construction material can comprise about 0.1 weight % of a supplemental material to about 0.5 weight % of a supplemental material, about 0.1 weight % of a supplemental material to about 1 weight % of a supplemental material, about 0.1 weight % of a supplemental material to about 2 weight % of a supplemental material, about 0.1 weight % of a supplemental material to about 3 weight % of a supplemental material, about 0.1 weight % of a supplemental material to about 4 weight % of a supplemental material, about 0.1 weight % of a supplemental material to about 5 weight % of a supplemental material, about 0.1 weight % of a supplemental material to about 10 weight % of a supplemental material, about 0.1 weight % of a supplemental material to about 15 weight % of a supplemental material, about 0.1 weight % of a supplemental material to about 20 weight % of a supplemental material, about 0.1 weight % of a supplemental material to about 30 weight % of a supplemental material, about 0.1 weight % of a supplemental material to about 40 weight % of a supplemental material, about 0.5 weight % of a supplemental material to about 1 weight % of a supplemental material, about 0.5 weight % of a supplemental material to about 2 weight % of a supplemental material, about 0.5 weight % of a supplemental material to about 3 weight % of a supplemental material, about 0.5 weight % of a supplemental material to about 4 weight % of a supplemental material, about 0.5 weight % of a supplemental material to about 5 weight % of a supplemental material, about 0.5 weight % of a supplemental material to about 10 weight % of a supplemental material, about 0.5 weight % of a supplemental material to about 15 weight % of a supplemental material, about 0.5 weight % of a supplemental material to about 20 weight % of a supplemental material, about 0.5 weight % of a supplemental material to about 30 weight % of a supplemental material, about 0.5 weight % of a supplemental material to about 40 weight % of a supplemental material, about 1 weight % of a supplemental material to about 2 weight % of a supplemental material, about 1 weight % of a supplemental material to about 3 weight % of a supplemental material, about 1 weight % of a supplemental material to about 4 weight % of a supplemental material, about 1 weight % of a supplemental material to about 5 weight % of a supplemental material, about 1 weight % of a supplemental material to about 10 weight % of a supplemental material, about 1 weight % of a supplemental material to about 15 weight % of a supplemental material, about 1 weight % of a supplemental material to about 20 weight % of a supplemental material, about 1 weight % of a supplemental material to about 30 weight % of a supplemental material, about 1 weight % of a supplemental material to about 40 weight % of a supplemental material, about 2 weight % of a supplemental material to about 3 weight % of a supplemental material, about 2 weight % of a supplemental material to about 4 weight % of a supplemental material, about 2 weight % of a supplemental material to about 5 weight % of a supplemental material, about 2 weight % of a supplemental material to about 10 weight % of a supplemental material, about 2 weight % of a supplemental material to about 15 weight % of a supplemental material, about 2 weight % of a supplemental material to about 20 weight % of a supplemental material, about 2 weight % of a supplemental material to about 30 weight % of a supplemental material, about 2 weight % of a supplemental material to about 40 weight % of a supplemental material, about 3 weight % of a supplemental material to about 4 weight % of a supplemental material, about 3 weight % of a supplemental material to about 5 weight % of a supplemental material, about 3 weight % of a supplemental material to about 10 weight % of a supplemental material, about 3 weight % of a supplemental material to about 15 weight % of a supplemental material, about 3 weight % of a supplemental material to about 20 weight % of a supplemental material, about 3 weight % of a supplemental material to about 30 weight % of a supplemental material, about 3 weight % of a supplemental material to about 40 weight % of a supplemental material, about 4 weight % of a supplemental material to about 5 weight % of a supplemental material, about 4 weight % of a supplemental material to about 10 weight % of a supplemental material, about 4 weight % of a supplemental material to about 15 weight % of a supplemental material, about 4 weight % of a supplemental material to about 20 weight % of a supplemental material, about 4 weight % of a supplemental material to about 30 weight % of a supplemental material, about 4 weight % of a supplemental material to about 40 weight % of a supplemental material, about 5 weight % of a supplemental material to about 10 weight % of a supplemental material, about 5 weight % of a supplemental material to about 15 weight % of a supplemental material, about 5 weight % of a supplemental material to about 20 weight % of a supplemental material, about 5 weight % of a supplemental material to about 30 weight % of a supplemental material, about 5 weight % of a supplemental material to about 40 weight % of a supplemental material, about 10 weight % of a supplemental material to about 15 weight % of a supplemental material, about 10 weight % of a supplemental material to about 20 weight % of a supplemental material, about 10 weight % of a supplemental material to about 30 weight % of a supplemental material, about 10 weight % of a supplemental material to about 40 weight % of a supplemental material, about 15 weight % of a supplemental material to about 20 weight % of a supplemental material, about 15 weight % of a supplemental material to about 30 weight % of a supplemental material, about 15 weight % of a supplemental material to about 40 weight % of a supplemental material, about 20 weight % of a supplemental material to about 30 weight % of a supplemental material, about 20 weight % of a supplemental material to about 40 weight % of a supplemental material, or about 30 weight % of a supplemental material to about 40 weight % of a supplemental material. In some embodiments, a bioconcrete construction material can comprise about 0.1 weight % of a supplemental material, about 0.5 weight % of a supplemental material, about 1 weight % of a supplemental material, about 2 weight % of a supplemental material, about 3 weight % of a supplemental material, about 4 weight % of a supplemental material, about 5 weight % of a supplemental material, about 10 weight % of a supplemental material, about 15 weight % of a supplemental material, about 20 weight % of a supplemental material, about 30 weight % of a supplemental material, or about 40 weight % of a supplemental material. In some embodiments, a bioconcrete construction material can comprise at least about 0.1 weight % of a supplemental material, about 0.5 weight % of a supplemental material, about 1 weight % of a supplemental material, about 2 weight % of a supplemental material, about 3 weight % of a supplemental material, about 4 weight % of a supplemental material, about 5 weight % of a supplemental material, about 10 weight % of a supplemental material, about 15 weight % of a supplemental material, about 20 weight % of a supplemental material, or about 30 weight % of a supplemental material. In some embodiments, a bioconcrete construction material can comprise at most about 0.5 weight % of a supplemental material, about 1 weight % of a supplemental material, about 2 weight % of a supplemental material, about 3 weight % of a supplemental material, about 4 weight % of a supplemental material, about 5 weight % of a supplemental material, about 10 weight % of a supplemental material, about 15 weight % of a supplemental material, about 20 weight % of a supplemental material, about 30 weight % of a supplemental material, or about 40 weight % of a supplemental material.

In some embodiments, a bioconcrete construction material has a compressive strength of about 900 psi to about 3,500 psi. In some embodiments, a bioconcrete construction material has a compressive strength of about 900 psi to about 1,000 psi, about 900 psi to about 1,100 psi, about 900 psi to about 1,200 psi, about 900 psi to about 1,300 psi, about 900 psi to about 1,400 psi, about 900 psi to about 1,600 psi, about 900 psi to about 1,800 psi, about 900 psi to about 2,000 psi, about 900 psi to about 2,500 psi, about 900 psi to about 3,000 psi, about 900 psi to about 3,500 psi, about 1,000 psi to about 1,100 psi, about 1,000 psi to about 1,200 psi, about 1,000 psi to about 1,300 psi, about 1,000 psi to about 1,400 psi, about 1,000 psi to about 1,600 psi, about 1,000 psi to about 1,800 psi, about 1,000 psi to about 2,000 psi, about 1,000 psi to about 2,500 psi, about 1,000 psi to about 3,000 psi, about 1,000 psi to about 3,500 psi, about 1,100 psi to about 1,200 psi, about 1,100 psi to about 1,300 psi, about 1,100 psi to about 1,400 psi, about 1,100 psi to about 1,600 psi, about 1,100 psi to about 1,800 psi, about 1,100 psi to about 2,000 psi, about 1,100 psi to about 2,500 psi, about 1,100 psi to about 3,000 psi, about 1,100 psi to about 3,500 psi, about 1,200 psi to about 1,300 psi, about 1,200 psi to about 1,400 psi, about 1,200 psi to about 1,600 psi, about 1,200 psi to about 1,800 psi, about 1,200 psi to about 2,000 psi, about 1,200 psi to about 2,500 psi, about 1,200 psi to about 3,000 psi, about 1,200 psi to about 3,500 psi, about 1,300 psi to about 1,400 psi, about 1,300 psi to about 1,600 psi, about 1,300 psi to about 1,800 psi, about 1,300 psi to about 2,000 psi, about 1,300 psi to about 2,500 psi, about 1,300 psi to about 3,000 psi, about 1,300 psi to about 3,500 psi, about 1,400 psi to about 1,600 psi, about 1,400 psi to about 1,800 psi, about 1,400 psi to about 2,000 psi, about 1,400 psi to about 2,500 psi, about 1,400 psi to about 3,000 psi, about 1,400 psi to about 3,500 psi, about 1,600 psi to about 1,800 psi, about 1,600 psi to about 2,000 psi, about 1,600 psi to about 2,500 psi, about 1,600 psi to about 3,000 psi, about 1,600 psi to about 3,500 psi, about 1,800 psi to about 2,000 psi, about 1,800 psi to about 2,500 psi, about 1,800 psi to about 3,000 psi, about 1,800 psi to about 3,500 psi, about 2,000 psi to about 2,500 psi, about 2,000 psi to about 3,000 psi, about 2,000 psi to about 3,500 psi, about 2,500 psi to about 3,000 psi, about 2,500 psi to about 3,500 psi, or about 3,000 psi to about 3,500 psi. In some embodiments, a bioconcrete construction material has a compressive strength of about 900 psi, about 1,000 psi, about 1,100 psi, about 1,200 psi, about 1,300 psi, about 1,400 psi, about 1,600 psi, about 1,800 psi, about 2,000 psi, about 2,500 psi, about 3,000 psi, or about 3,500 psi. In some embodiments, a bioconcrete construction material has a compressive strength of at least about 900 psi, about 1,000 psi, about 1,100 psi, about 1,200 psi, about 1,300 psi, about 1,400 psi, about 1,600 psi, about 1,800 psi, about 2,000 psi, about 2,500 psi, or about 3,000 psi. In some embodiments, a bioconcrete construction material has a compressive strength of at most about 1,000 psi, about 1,100 psi, about 1,200 psi, about 1,300 psi, about 1,400 psi, about 1,600 psi, about 1,800 psi, about 2,000 psi, about 2,500 psi, about 3,000 psi, or about 3,500 psi.

In some embodiments, the mass ratio of a biocement to a supplemental material in a bioconcrete is about 1:1 to about 50:1. In some embodiments, the mass ratio of a biocement to a supplemental material in a bioconcrete is about 1:1 to about 2:1, about 1:1 to about 3:1, about 1:1 to about 4:1, about 1:1 to about 5:1, about 1:1 to about 6:1, about 1:1 to about 7:1, about 1:1 to about 8:1, about 1:1 to about 9:1, about 1:1 to about 10:1, about 1:1 to about 20:1, about 1:1 to about 50:1, about 2:1 to about 3:1, about 2:1 to about 4:1, about 2:1 to about 5:1, about 2:1 to about 6:1, about 2:1 to about 7:1, about 2:1 to about 8:1, about 2:1 to about 9:1, about 2:1 to about 10:1, about 2:1 to about 20:1, about 2:1 to about 50:1, about 3:1 to about 4:1, about 3:1 to about 5:1, about 3:1 to about 6:1, about 3:1 to about 7:1, about 3:1 to about 8:1, about 3:1 to about 9:1, about 3:1 to about 10:1, about 3:1 to about 20:1, about 3:1 to about 50:1, about 4:1 to about 5:1, about 4:1 to about 6:1, about 4:1 to about 7:1, about 4:1 to about 8:1, about 4:1 to about 9:1, about 4:1 to about 10:1, about 4:1 to about 20:1, about 4:1 to about 50:1, about 5:1 to about 6:1, about 5:1 to about 7:1, about 5:1 to about 8:1, about 5:1 to about 9:1, about 5:1 to about 10:1, about 5:1 to about 20:1, about 5:1 to about 50:1, about 6:1 to about 7:1, about 6:1 to about 8:1, about 6:1 to about 9:1, about 6:1 to about 10:1, about 6:1 to about 20:1, about 6:1 to about 50:1, about 7:1 to about 8:1, about 7:1 to about 9:1, about 7:1 to about 10:1, about 7:1 to about 20:1, about 7:1 to about 50:1, about 8:1 to about 9:1, about 8:1 to about 10:1, about 8:1 to about 20:1, about 8:1 to about 50:1, about 9:1 to about 10:1, about 9:1 to about 20:1, about 9:1 to about 50:1, about 10:1 to about 20:1, about 10:1 to about 50:1, or about 20:1 to about 50:1. In some embodiments, the mass ratio of a biocement to a supplemental material in a bioconcrete is about 1:1, about 2:1, about 3:1, about 4:1, about 5:1, about 6:1, about 7:1, about 8:1, about 9:1, about 10:1, about 20:1, or about 50:1. In some embodiments, the mass ratio of a biocement to a supplemental material in a bioconcrete is at least about 1:1, about 2:1, about 3:1, about 4:1, about 5:1, about 6:1, about 7:1, about 8:1, about 9:1, about 10:1, or about 20:1. In some embodiments, the mass ratio of a biocement to a supplemental material in a bioconcrete is at most about 2:1, about 3:1, about 4:1, about 5:1, about 6:1, about 7:1, about 8:1, about 9:1, about 10:1, about 20:1, or about 50:1.

In some embodiments, the mass ratio of a calcium carbonate to a supplemental material in a bioconcrete is about 1:1 to about 50:1. In some embodiments, the mass ratio of a calcium carbonate to a supplemental material in a bioconcrete is about 1:1 to about 2:1, about 1:1 to about 3:1, about 1:1 to about 4:1, about 1:1 to about 5:1, about 1:1 to about 6:1, about 1:1 to about 7:1, about 1:1 to about 8:1, about 1:1 to about 9:1, about 1:1 to about 10:1, about 1:1 to about 20:1, about 1:1 to about 50:1, about 2:1 to about 3:1, about 2:1 to about 4:1, about 2:1 to about 5:1, about 2:1 to about 6:1, about 2:1 to about 7:1, about 2:1 to about 8:1, about 2:1 to about 9:1, about 2:1 to about 10:1, about 2:1 to about 20:1, about 2:1 to about 50:1, about 3:1 to about 4:1, about 3:1 to about 5:1, about 3:1 to about 6:1, about 3:1 to about 7:1, about 3:1 to about 8:1, about 3:1 to about 9:1, about 3:1 to about 10:1, about 3:1 to about 20:1, about 3:1 to about 50:1, about 4:1 to about 5:1, about 4:1 to about 6:1, about 4:1 to about 7:1, about 4:1 to about 8:1, about 4:1 to about 9:1, about 4:1 to about 10:1, about 4:1 to about 20:1, about 4:1 to about 50:1, about 5:1 to about 6:1, about 5:1 to about 7:1, about 5:1 to about 8:1, about 5:1 to about 9:1, about 5:1 to about 10:1, about 5:1 to about 20:1, about 5:1 to about 50:1, about 6:1 to about 7:1, about 6:1 to about 8:1, about 6:1 to about 9:1, about 6:1 to about 10:1, about 6:1 to about 20:1, about 6:1 to about 50:1, about 7:1 to about 8:1, about 7:1 to about 9:1, about 7:1 to about 10:1, about 7:1 to about 20:1, about 7:1 to about 50:1, about 8:1 to about 9:1, about 8:1 to about 10:1, about 8:1 to about 20:1, about 8:1 to about 50:1, about 9:1 to about 10:1, about 9:1 to about 20:1, about 9:1 to about 50:1, about 10:1 to about 20:1, about 10:1 to about 50:1, or about 20:1 to about 50:1. In some embodiments, the mass ratio of a calcium carbonate to a supplemental material in a bioconcrete is about 1:1, about 2:1, about 3:1, about 4:1, about 5:1, about 6:1, about 7:1, about 8:1, about 9:1, about 10:1, about 20:1, or about 50:1. In some embodiments, the mass ratio of a calcium carbonate to a supplemental material in a bioconcrete is at least about 1:1, about 2:1, about 3:1, about 4:1, about 5:1, about 6:1, about 7:1, about 8:1, about 9:1, about 10:1, or about 20:1. In some embodiments, the mass ratio of a calcium carbonate to a supplemental material in a bioconcrete is at most about 2:1, about 3:1, about 4:1, about 5:1, about 6:1, about 7:1, about 8:1, about 9:1, about 10:1, about 20:1, or about 50:1.

In some embodiments, the construction material has a carbon footprint of at least about 10% less than a functionally equivalent construction material made from ACI 318 structural concrete to about 99% less than a functionally equivalent construction material made from ACI 318 structural concrete. In some embodiments, the construction material has a carbon footprint of at least about 10% less than a functionally equivalent construction material made from ACI 318 structural concrete to about 20% less than a functionally equivalent construction material made from ACI 318 structural concrete, about 10% less than a functionally equivalent construction material made from ACI 318 structural concrete to about 30% less than a functionally equivalent construction material made from ACI 318 structural concrete, about 10% less than a functionally equivalent construction material made from ACI 318 structural concrete to about 40% less than a functionally equivalent construction material made from ACI 318 structural concrete, about 10% less than a functionally equivalent construction material made from ACI 318 structural concrete to about 50% less than a functionally equivalent construction material made from ACI 318 structural concrete, about 10% less than a functionally equivalent construction material made from ACI 318 structural concrete to about 60% less than a functionally equivalent construction material made from ACI 318 structural concrete, about 10% less than a functionally equivalent construction material made from ACI 318 structural concrete to about 70% less than a functionally equivalent construction material made from ACI 318 structural concrete, about 10% less than a functionally equivalent construction material made from ACI 318 structural concrete to about 80% less than a functionally equivalent construction material made from ACI 318 structural concrete, about 10% less than a functionally equivalent construction material made from ACI 318 structural concrete to about 90% less than a functionally equivalent construction material made from ACI 318 structural concrete, about 10% less than a functionally equivalent construction material made from ACI 318 structural concrete to about 95% less than a functionally equivalent construction material made from ACI 318 structural concrete, about 10% less than a functionally equivalent construction material made from ACI 318 structural concrete to about 99% less than a functionally equivalent construction material made from ACI 318 structural concrete, about 20% less than a functionally equivalent construction material made from ACI 318 structural concrete to about 30% less than a functionally equivalent construction material made from ACI 318 structural concrete, about 20% less than a functionally equivalent construction material made from ACI 318 structural concrete to about 40% less than a functionally equivalent construction material made from ACI 318 structural concrete, about 20% less than a functionally equivalent construction material made from ACI 318 structural concrete to about 50% less than a functionally equivalent construction material made from ACI 318 structural concrete, about 20% less than a functionally equivalent construction material made from ACI 318 structural concrete to about 60% less than a functionally equivalent construction material made from ACI 318 structural concrete, about 20% less than a functionally equivalent construction material made from ACI 318 structural concrete to about 70% less than a functionally equivalent construction material made from ACI 318 structural concrete, about 20% less than a functionally equivalent construction material made from ACI 318 structural concrete to about 80% less than a functionally equivalent construction material made from ACI 318 structural concrete, about 20% less than a functionally equivalent construction material made from ACI 318 structural concrete to about 90% less than a functionally equivalent construction material made from ACI 318 structural concrete, about 20% less than a functionally equivalent construction material made from ACI 318 structural concrete to about 95% less than a functionally equivalent construction material made from ACI 318 structural concrete, about 20% less than a functionally equivalent construction material made from ACI 318 structural concrete to about 99% less than a functionally equivalent construction material made from ACI 318 structural concrete, about 30% less than a functionally equivalent construction material made from ACI 318 structural concrete to about 40% less than a functionally equivalent construction material made from ACI 318 structural concrete, about 30% less than a functionally equivalent construction material made from ACI 318 structural concrete to about 50% less than a functionally equivalent construction material made from ACI 318 structural concrete, about 30% less than a functionally equivalent construction material made from ACI 318 structural concrete to about 60% less than a functionally equivalent construction material made from ACI 318 structural concrete, about 30% less than a functionally equivalent construction material made from ACI 318 structural concrete to about 70% less than a functionally equivalent construction material made from ACI 318 structural concrete, about 30% less than a functionally equivalent construction material made from ACI 318 structural concrete to about 80% less than a functionally equivalent construction material made from ACI 318 structural concrete, about 30% less than a functionally equivalent construction material made from ACI 318 structural concrete to about 90% less than a functionally equivalent construction material made from ACI 318 structural concrete, about 30% less than a functionally equivalent construction material made from ACI 318 structural concrete to about 95% less than a functionally equivalent construction material made from ACI 318 structural concrete, about 30% less than a functionally equivalent construction material made from ACI 318 structural concrete to about 99% less than a functionally equivalent construction material made from ACI 318 structural concrete, about 40% less than a functionally equivalent construction material made from ACI 318 structural concrete to about 50% less than a functionally equivalent construction material made from ACI 318 structural concrete, about 40% less than a functionally equivalent construction material made from ACI 318 structural concrete to about 60% less than a functionally equivalent construction material made from ACI 318 structural concrete, about 40% less than a functionally equivalent construction material made from ACI 318 structural concrete to about 70% less than a functionally equivalent construction material made from ACI 318 structural concrete, about 40% less than a functionally equivalent construction material made from ACI 318 structural concrete to about 80% less than a functionally equivalent construction material made from ACI 318 structural concrete, about 40% less than a functionally equivalent construction material made from ACI 318 structural concrete to about 90% less than a functionally equivalent construction material made from ACI 318 structural concrete, about 40% less than a functionally equivalent construction material made from ACI 318 structural concrete to about 95% less than a functionally equivalent construction material made from ACI 318 structural concrete, about 40% less than a functionally equivalent construction material made from ACI 318 structural concrete to about 99% less than a functionally equivalent construction material made from ACI 318 structural concrete, about 50% less than a functionally equivalent construction material made from ACI 318 structural concrete to about 60% less than a functionally equivalent construction material made from ACI 318 structural concrete, about 50% less than a functionally equivalent construction material made from ACI 318 structural concrete to about 70% less than a functionally equivalent construction material made from ACI 318 structural concrete, about 50% less than a functionally equivalent construction material made from ACI 318 structural concrete to about 80% less than a functionally equivalent construction material made from ACI 318 structural concrete, about 50% less than a functionally equivalent construction material made from ACI 318 structural concrete to about 90% less than a functionally equivalent construction material made from ACI 318 structural concrete, about 50% less than a functionally equivalent construction material made from ACI 318 structural concrete to about 95% less than a functionally equivalent construction material made from ACI 318 structural concrete, about 50% less than a functionally equivalent construction material made from ACI 318 structural concrete to about 99% less than a functionally equivalent construction material made from ACI 318 structural concrete, about 60% less than a functionally equivalent construction material made from ACI 318 structural concrete to about 70% less than a functionally equivalent construction material made from ACI 318 structural concrete, about 60% less than a functionally equivalent construction material made from ACI 318 structural concrete to about 80% less than a functionally equivalent construction material made from ACI 318 structural concrete, about 60% less than a functionally equivalent construction material made from ACI 318 structural concrete to about 90% less than a functionally equivalent construction material made from ACI 318 structural concrete, about 60% less than a functionally equivalent construction material made from ACI 318 structural concrete to about 95% less than a functionally equivalent construction material made from ACI 318 structural concrete, about 60% less than a functionally equivalent construction material made from ACI 318 structural concrete to about 99% less than a functionally equivalent construction material made from ACI 318 structural concrete, about 70% less than a functionally equivalent construction material made from ACI 318 structural concrete to about 80% less than a functionally equivalent construction material made from ACI 318 structural concrete, about 70% less than a functionally equivalent construction material made from ACI 318 structural concrete to about 90% less than a functionally equivalent construction material made from ACI 318 structural concrete, about 70% less than a functionally equivalent construction material made from ACI 318 structural concrete to about 95% less than a functionally equivalent construction material made from ACI 318 structural concrete, about 70% less than a functionally equivalent construction material made from ACI 318 structural concrete to about 99% less than a functionally equivalent construction material made from ACI 318 structural concrete, about 80% less than a functionally equivalent construction material made from ACI 318 structural concrete to about 90% less than a functionally equivalent construction material made from ACI 318 structural concrete, about 80% less than a functionally equivalent construction material made from ACI 318 structural concrete to about 95% less than a functionally equivalent construction material made from ACI 318 structural concrete, about 80% less than a functionally equivalent construction material made from ACI 318 structural concrete to about 99% less than a functionally equivalent construction material made from ACI 318 structural concrete, about 90% less than a functionally equivalent construction material made from ACI 318 structural concrete to about 95% less than a functionally equivalent construction material made from ACI 318 structural concrete, about 90% less than a functionally equivalent construction material made from ACI 318 structural concrete to about 99% less than a functionally equivalent construction material made from ACI 318 structural concrete, or about 95% less than a functionally equivalent construction material made from ACI 318 structural concrete to about 99% less than a functionally equivalent construction material made from ACI 318 structural concrete. In some embodiments, the construction material has a carbon footprint of at least about 10% less than a functionally equivalent construction material made from ACI 318 structural concrete, about 20% less than a functionally equivalent construction material made from ACI 318 structural concrete, about 30% less than a functionally equivalent construction material made from ACI 318 structural concrete, about 40% less than a functionally equivalent construction material made from ACI 318 structural concrete, about 50% less than a functionally equivalent construction material made from ACI 318 structural concrete, about 60% less than a functionally equivalent construction material made from ACI 318 structural concrete, about 70% less than a functionally equivalent construction material made from ACI 318 structural concrete, about 80% less than a functionally equivalent construction material made from ACI 318 structural concrete, about 90% less than a functionally equivalent construction material made from ACI 318 structural concrete, about 95% less than a functionally equivalent construction material made from ACI 318 structural concrete, or about 99% less than a functionally equivalent construction material made from ACI 318 structural concrete. In some embodiments, the construction material has a carbon footprint of at least at least about 10% less than a functionally equivalent construction material made from ACI 318 structural concrete, about 20% less than a functionally equivalent construction material made from ACI 318 structural concrete, about 30% less than a functionally equivalent construction material made from ACI 318 structural concrete, about 40% less than a functionally equivalent construction material made from ACI 318 structural concrete, about 50% less than a functionally equivalent construction material made from ACI 318 structural concrete, about 60% less than a functionally equivalent construction material made from ACI 318 structural concrete, about 70% less than a functionally equivalent construction material made from ACI 318 structural concrete, about 80% less than a functionally equivalent construction material made from ACI 318 structural concrete, about 90% less than a functionally equivalent construction material made from ACI 318 structural concrete, or about 95% less than a functionally equivalent construction material made from ACI 318 structural concrete. In some embodiments, the construction material has a carbon footprint of at least at most about 20% less than a functionally equivalent construction material made from ACI 318 structural concrete, about 30% less than a functionally equivalent construction material made from ACI 318 structural concrete, about 40% less than a functionally equivalent construction material made from ACI 318 structural concrete, about 50% less than a functionally equivalent construction material made from ACI 318 structural concrete, about 60% less than a functionally equivalent construction material made from ACI 318 structural concrete, about 70% less than a functionally equivalent construction material made from ACI 318 structural concrete, about 80% less than a functionally equivalent construction material made from ACI 318 structural concrete, about 90% less than a functionally equivalent construction material made from ACI 318 structural concrete, about 95% less than a functionally equivalent construction material made from ACI 318 structural concrete, or about 99% less than a functionally equivalent construction material made from ACI 318 structural concrete.

In some embodiments, the finished bioconcrete construction material comprises about 0.5 weight % biocement to about 25 weight % biocement. In some embodiments, the finished bioconcrete construction material comprises about 0.5 weight % biocement to about 1 weight % biocement, about 0.5 weight % biocement to about 2 weight % biocement, about 0.5 weight % biocement to about 3 weight % biocement, about 0.5 weight % biocement to about 5 weight % biocement, about 0.5 weight % biocement to about 7 weight % biocement, about 0.5 weight % biocement to about 9 weight % biocement, about 0.5 weight % biocement to about 10 weight % biocement, about 0.5 weight % biocement to about 12 weight % biocement, about 0.5 weight % biocement to about 16 weight % biocement, about 0.5 weight % biocement to about 18 weight % biocement, about 0.5 weight % biocement to about 25 weight % biocement, about 1 weight % biocement to about 2 weight % biocement, about 1 weight % biocement to about 3 weight % biocement, about 1 weight % biocement to about 5 weight % biocement, about 1 weight % biocement to about 7 weight % biocement, about 1 weight % biocement to about 9 weight % biocement, about 1 weight % biocement to about 10 weight % biocement, about 1 weight % biocement to about 12 weight % biocement, about 1 weight % biocement to about 16 weight % biocement, about 1 weight % biocement to about 18 weight % biocement, about 1 weight % biocement to about 25 weight % biocement, about 2 weight % biocement to about 3 weight % biocement, about 2 weight % biocement to about 5 weight % biocement, about 2 weight % biocement to about 7 weight % biocement, about 2 weight % biocement to about 9 weight % biocement, about 2 weight % biocement to about 10 weight % biocement, about 2 weight % biocement to about 12 weight % biocement, about 2 weight % biocement to about 16 weight % biocement, about 2 weight % biocement to about 18 weight % biocement, about 2 weight % biocement to about 25 weight % biocement, about 3 weight % biocement to about 5 weight % biocement, about 3 weight % biocement to about 7 weight % biocement, about 3 weight % biocement to about 9 weight % biocement, about 3 weight % biocement to about 10 weight % biocement, about 3 weight % biocement to about 12 weight % biocement, about 3 weight % biocement to about 16 weight % biocement, about 3 weight % biocement to about 18 weight % biocement, about 3 weight % biocement to about 25 weight % biocement, about 5 weight % biocement to about 7 weight % biocement, about 5 weight % biocement to about 9 weight % biocement, about 5 weight % biocement to about 10 weight % biocement, about 5 weight % biocement to about 12 weight % biocement, about 5 weight % biocement to about 16 weight % biocement, about 5 weight % biocement to about 18 weight % biocement, about 5 weight % biocement to about 25 weight % biocement, about 7 weight % biocement to about 9 weight % biocement, about 7 weight % biocement to about 10 weight % biocement, about 7 weight % biocement to about 12 weight % biocement, about 7 weight % biocement to about 16 weight % biocement, about 7 weight % biocement to about 18 weight % biocement, about 7 weight % biocement to about 25 weight % biocement, about 9 weight % biocement to about 10 weight % biocement, about 9 weight % biocement to about 12 weight % biocement, about 9 weight % biocement to about 16 weight % biocement, about 9 weight % biocement to about 18 weight % biocement, about 9 weight % biocement to about 25 weight % biocement, about 10 weight % biocement to about 12 weight % biocement, about 10 weight % biocement to about 16 weight % biocement, about 10 weight % biocement to about 18 weight % biocement, about 10 weight % biocement to about 25 weight % biocement, about 12 weight % biocement to about 16 weight % biocement, about 12 weight % biocement to about 18 weight % biocement, about 12 weight % biocement to about 25 weight % biocement, about 16 weight % biocement to about 18 weight % biocement, about 16 weight % biocement to about 25 weight % biocement, or about 18 weight % biocement to about 25 weight % biocement. In some embodiments, the finished bioconcrete construction material comprises about 0.5 weight % biocement, about 1 weight % biocement, about 2 weight % biocement, about 3 weight % biocement, about 5 weight % biocement, about 7 weight % biocement, about 9 weight % biocement, about 10 weight % biocement, about 12 weight % biocement, about 16 weight % biocement, about 18 weight % biocement, or about 25 weight % biocement. In some embodiments, the finished bioconcrete construction material comprises at least about 0.5 weight % biocement, about 1 weight % biocement, about 2 weight % biocement, about 3 weight % biocement, about 5 weight % biocement, about 7 weight % biocement, about 9 weight % biocement, about 10 weight % biocement, about 12 weight % biocement, about 16 weight % biocement, or about 18 weight % biocement. In some embodiments, the finished bioconcrete construction material comprises at most about 1 weight % biocement, about 2 weight % biocement, about 3 weight % biocement, about 5 weight % biocement, about 7 weight % biocement, about 9 weight % biocement, about 10 weight % biocement, about 12 weight % biocement, about 16 weight % biocement, about 18 weight % biocement, or about 25 weight % biocement.

In some embodiments, the mass of calcium carbonate in a bioconcrete construction material is about 0.01 weight % to about 20 weight %. In some embodiments, the mass of calcium carbonate in a bioconcrete construction material is about 0.01 weight % to about 0.1 weight %, about 0.01 weight % to about 0.5 weight %, about 0.01 weight % to about 1 weight %, about 0.01 weight % to about 2 weight %, about 0.01 weight % to about 3 weight %, about 0.01 weight % to about 4 weight %, about 0.01 weight % to about 5 weight %, about 0.01 weight % to about 7 weight %, about 0.01 weight % to about 10 weight %, about 0.01 weight % to about 15 weight %, about 0.01 weight % to about 20 weight %, about 0.1 weight % to about 0.5 weight %, about 0.1 weight % to about 1 weight %, about 0.1 weight % to about 2 weight %, about 0.1 weight % to about 3 weight %, about 0.1 weight % to about 4 weight %, about 0.1 weight % to about 5 weight %, about 0.1 weight % to about 7 weight %, about 0.1 weight % to about 10 weight %, about 0.1 weight % to about 15 weight %, about 0.1 weight % to about 20 weight %, about 0.5 weight % to about 1 weight %, about 0.5 weight % to about 2 weight %, about 0.5 weight % to about 3 weight %, about 0.5 weight % to about 4 weight %, about 0.5 weight % to about 5 weight %, about 0.5 weight % to about 7 weight %, about 0.5 weight % to about 10 weight %, about 0.5 weight % to about 15 weight %, about 0.5 weight % to about 20 weight %, about 1 weight % to about 2 weight %, about 1 weight % to about 3 weight %, about 1 weight % to about 4 weight %, about 1 weight % to about 5 weight %, about 1 weight % to about 7 weight %, about 1 weight % to about 10 weight %, about 1 weight % to about 15 weight %, about 1 weight % to about 20 weight %, about 2 weight % to about 3 weight %, about 2 weight % to about 4 weight %, about 2 weight % to about 5 weight %, about 2 weight % to about 7 weight %, about 2 weight % to about 10 weight %, about 2 weight % to about 15 weight %, about 2 weight % to about 20 weight %, about 3 weight % to about 4 weight %, about 3 weight % to about 5 weight %, about 3 weight % to about 7 weight %, about 3 weight % to about 10 weight %, about 3 weight % to about 15 weight %, about 3 weight % to about 20 weight %, about 4 weight % to about 5 weight %, about 4 weight % to about 7 weight %, about 4 weight % to about 10 weight %, about 4 weight % to about 15 weight %, about 4 weight % to about 20 weight %, about 5 weight % to about 7 weight %, about 5 weight % to about 10 weight %, about 5 weight % to about 15 weight %, about 5 weight % to about 20 weight %, about 7 weight % to about 10 weight %, about 7 weight % to about 15 weight %, about 7 weight % to about 20 weight %, about 10 weight % to about 15 weight %, about 10 weight % to about 20 weight %, or about 15 weight % to about 20 weight %. In some embodiments, the mass of calcium carbonate in a bioconcrete construction material is about 0.01 weight %, about 0.1 weight %, about 0.5 weight %, about 1 weight %, about 2 weight %, about 3 weight %, about 4 weight %, about 5 weight %, about 7 weight %, about 10 weight %, about 15 weight %, or about 20 weight %. In some embodiments, the mass of calcium carbonate in a bioconcrete construction material is at least about 0.01 weight %, about 0.1 weight %, about 0.5 weight %, about 1 weight %, about 2 weight %, about 3 weight %, about 4 weight %, about 5 weight %, about 7 weight %, about 10 weight %, or about 15 weight %. In some embodiments, the mass of calcium carbonate in a bioconcrete construction material is at most about 0.1 weight %, about 0.5 weight %, about 1 weight %, about 2 weight %, about 3 weight %, about 4 weight %, about 5 weight %, about 7 weight %, about 10 weight %, about 15 weight %, or about 20 weight %.

### Methods of Producing Biocement Compositions Comprising a Supplemental Material

Methods of manufacturing described herein can be used to make any of the construction materials described herein. Methods can comprise combining aggregate particles with biocementation reagents and a supplemental material to yield a biocement mixture. Method can comprise reacting the cementation reagents in the presence of the supplemental material to produce a biocement, thereby yielding a construction material. The steps of the method can be carried out in a temperature-controlled and humidity-controlled environment.

Roughening of aggregate particles can be performed by mixing particles together in a mixer or blender with sufficient force to create cracks in and crevices on particle surfaces, by adding ball bearings or another substance to the aggregate particles with a hardness equal to or greater than the particles themselves, by passing particles over a roughening agent such as, for example, sand, steel, or industrial diamonds, or another roughening agent known to those skilled in the art or combinations thereof.

A temperature, humidity, and/or pH of a bioconcrete mixture can monitored and controlled during the reaction that forms the biocement. Biocementation reagents comprise at least one biological organism or enzyme further comprising urease or cells of a urease-producing microorganism. Biocementation reagents comprise an acid-producing enzyme or cells of an acid-producing microorganism and/or carbonic anhydrase or cells of a carbonic anhydrase-producing microorganism.

Uricase-producing microorganisms can comprise but are not limited to Sporosarcina pasteurii, Sporosarcina ureae, Proteus vulgaris, Bacillus sphaericus, Myxococcus xanthus, Proteus mirabilis, Bacillus megaterium, Helicobacter pylori, and combinations of two or more thereof. In some embodiments, the cells comprise spores. Biocementation reagents can further comprise nutrients which promote the growth or enzymatic activities of microorganisms. In some embodiments, the nutrients comprise one or more of salts, amino acids, proteins, peptides, carbohydrates, saccharides, polysaccharides, fatty acids, oil, vitamins and minerals.

Biocementation reagents can comprise a calcium source. Biocementation reagents can comprise a urea source. Biocementation reagents comprise calcium carbonate. Biocementation reagents comprise calcium chloride. Biocementation reagents can comprise cells of a urea-producing microorganism. In some embodiments, the urea-producing microorganism is selected from the group of: Pseudomonas, Delaya avenusta, Thiosphaera pantotropha, Pseudomonas stutzen, Fragilaria crotonensis, Pseudoalteromonas spp., Pseudoalteromonas haloplanktis, Halomonas venusta, Pseudomonas balearica, Pseudomonas stutzeri, Bacillus megaterium, Exiguobacterium aurantiacum, Pseudoalteromonas aliena, Pseudoalteromonas luteoviolacea, E. coli, and variants, serotypes, mutations, recombinant forms, and combinations thereof. In some embodiments, the acid-producing microorganism is selected from the group consisting of: Variovorax, Klebsiella, Pseudomonas, Bacillus, Exiguobacterium, Microbacterium, Curtobacterium, Rathayibacter, CellFimi2, Streptomyces, Raoultella, B. pumilus, B. safanensis, B. simplex, B. licheniformis, and combinations thereof.

In some embodiments, the acid produced by the acid-producing enzyme of the cells of the acid-producing microorganism is a carboxylic acid. In some embodiments, the acid produced by the acid-producing enzyme of the cells of the acid-producing microorganism is acetic acid, formic acid, propionic acid, butyric acid, gluconic acid, succinic acid, lactic acid, or citric acid. In some embodiments, the method further comprises compacting the mixture to reduce the average linear distance between adjacent aggregate particles by at least about 25%. In some embodiments, the method further comprises compacting the mixture to produce an absolute packing efficiency of the aggregate particles of at least about 50%.

In some embodiments, the method further comprises compacting the mixture by placing the mixture in a vibratory press and applying pressure and vibration to reduce the volume of empty space between a plurality of biocement coated aggregate particles. In some embodiments, the vibratory motor of the press is operated at a rotational speed from about 100 RPM to about 7200 RPM. In some embodiments, the vibratory motor of the press is operated at a duty cycle from 0.01% to about 100%. In some embodiments, the vibration and pressure are applied simultaneously. In some embodiments, the vibration and pressure are applied alternatively. In some embodiments, the finished construction material comprises at least about 2% biocement by weight. In some embodiments, the finished construction material comprises at most about 20% biocement by weight.

### Computer Systems

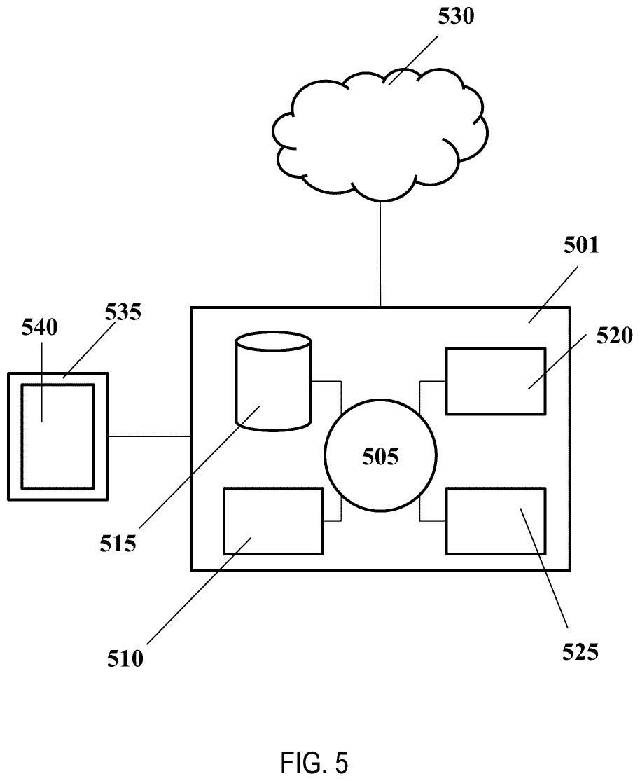

The present disclosure provides computer systems that are programmed to implement methods of the disclosure. FIG. 5 shows a computer system 501 that is programmed or otherwise configured to control a biocement mixing device. The computer system 501 can regulate various aspects of biocement mixing device of the present disclosure, such as, for example, control of the temperature, humidity, pH, mixing speed, or other controllable features as described above. The computer system 501 can be an electronic device of a user or a computer system that is remotely located with respect to the electronic device. The electronic device can be a mobile electronic device.

The computer system 501 includes a central processing unit (CPU, also “processor” and “computer processor” herein) 505, which can be a single core or multi core processor, or a plurality of processors for parallel processing. The computer system 501 also includes memory or memory location 510 (e.g., random-access memory, read-only memory, flash memory), electronic storage unit 515 (e.g., hard disk), communication interface 520 (e.g., network adapter) for communicating with one or more other systems, and peripheral devices 525, such as cache, other memory, data storage and/or electronic display adapters. The memory 510, storage unit 515, interface 520 and peripheral devices 525 are in communication with the CPU 505 through a communication bus (solid lines), such as a motherboard. The storage unit 515 can be a data storage unit (or data repository) for storing data. The computer system 501 can be operatively coupled to a computer network (“network”) 530 with the aid of the communication interface 520. The network 530 can be the Internet, an internet and/or extranet, or an intranet and/or extranet that is in communication with the Internet. The network 530 in some cases is a telecommunication and/or data network. The network 530 can include one or more computer servers, which can enable distributed computing, such as cloud computing. The network 530, in some cases with the aid of the computer system 501, can implement a peer-to-peer network, which may enable devices coupled to the computer system 501 to behave as a client or a server.

The CPU 505 can execute a sequence of machine-readable instructions, which can be embodied in a program or software. The instructions may be stored in a memory location, such as the memory 510. The instructions can be directed to the CPU 505, which can subsequently program or otherwise configure the CPU 505 to implement methods of the present disclosure. Examples of operations performed by the CPU 505 can include fetch, decode, execute, and writeback.

The CPU 505 can be part of a circuit, such as an integrated circuit. One or more other components of the system 501 can be included in the circuit. In some cases, the circuit is an application specific integrated circuit (ASIC).

The storage unit 515 can store files, such as drivers, libraries and saved programs. The storage unit 515 can store user data, e.g., user preferences and user programs. The computer system 501 in some cases can include one or more additional data storage units that are external to the computer system 501, such as located on a remote server that is in communication with the computer system 501 through an intranet or the Internet.

The computer system 501 can communicate with one or more remote computer systems through the network 530. For instance, the computer system 501 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 501 via the network 530.

Methods as described herein can be implemented by way of machine (e.g., computer processor) executable code stored on an electronic storage location of the computer system 501, such as, for example, on the memory 510 or electronic storage unit 515. The machine executable or machine readable code can be provided in the form of software. During use, the code can be executed by the processor 505. In some cases, the code can be retrieved from the storage unit 515 and stored on the memory 510 for ready access by the processor 505. In some situations, the electronic storage unit 515 can be precluded, and machine-executable instructions are stored on memory 510.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 501 can include or be in communication with an electronic display 535 that comprises a user interface (UI) 540 for providing, for example, programming interfaces for mixing time, reagent dose and timing, reaction temperatures, mixing speed, duty cycle, etc. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 505. The algorithm can, for example, control the reaction temperature or adapt reaction conditions to sensor data.

### EXAMPLES

### Example 1—Production of a High-Strength Bioconcrete

A press formed bioconcrete construction material was made by adding a manufactured granite sand aggregate mixture with particle size fractions of 50% Mesh 8/16, 25% Mesh 50/100, and 25% fine particles under Mesh 100 to a prototype biocement mixing device constructed of 304 stainless steel with a 0.01 yd³ mixing volume. The initial cementation reaction was performed over 48 hours, over which time the mixer added 18 L/kg of reagent solution in evenly divided 5 minute intervals at a controlled temperature range of 30° C. to 34° C. The moisture content during mixing was held in the range of 5-15%.

The reagents for the initial cementation reaction contained urease-producing microorganisms (Sporosarcina pasteurii) in a concentration range of 1e8 to 1e10 CFU/mL, a calcium concentration of 0.1-3M, a urea concentration of 0.1-3M, and nutrients which promote the urease-activity of the microorganisms. The mixer was operated at 60 RPM for 10 seconds once every 4 hours.

The initial cementation reaction produced an estimate 50-100 μm thick coating of calcium carbonate biocement around the aggregate particles.

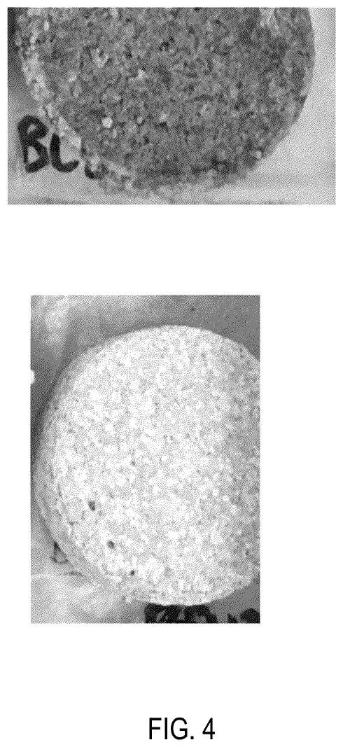

Secondary cementation reagents were then mixed with the coated aggregate. The secondary cementation reagents contained a calcium chloride concentration between 1M and 1.5M and a urea concentration between 1 and 1.5M. The mixture was then transferred to a vibratory press which was operated 20° C. and vibration was applied by use of a motor running at 2840 RPM. A press force of 0.16 tons/in² was applied hydraulically. Pressed materials were placed in a temperature and humidity controlled environment where the final bonding biocement bridges were built over a period of 24 hours, consolidating the coated aggregate material into the solid product depicted in FIG. 4, which had a compressive strength of 810 psi.

### Example 2—High-Strength Bioconcrete Compositions Comprising Ordinary Portland Cement (OPC) Supplemental Material

Samples of exemplary bioconcrete compositions were prepared by mixing aggregate, calcium carbonate, Ordinary Portland cement, and an aqueous solution containing biocementation reagents. For samples listed in Table 1 as “Biological system”, a combination of an acid producing bacteria and a calcium carbonate precipitating bacteria were further added to the mixture. Mixtures were then compacted in a vibratory press and cured for 3 to 7 days at 30° C. and 90% relative humidity. Compositions and compressive strengths of three representative blends with and without the addition of bacteria are shown in Table 1.

TABLE 1
Compressive strength of construction materials comprising
aggregate, calcium carbonate, and a supplemental material

| Sample Identifier | | A | B | C |
|---|---|---|---|---|
| Compressive | Biological | 2285 | 3621 | 3761 |
| strength (PSI) | system | | | |
| | No biology | 1519 | 1224 | 910 |
| | % improvement | 150 | 295 | 413 |
| Dry components | Aggregate % | 86 | 86 | 86 |
| | CaCO₃% | 12 | 12 | 12 |
| | Supplemental | 1.3 | 1.3 | 1.3 |
| | material % | | | |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

What is claimed is:

1. A method of producing a bioconcrete construction material, the method comprising:
combining aggregate particles with a first measured dose of at least one biological organism or enzyme and a first measured dose of cementation reagents in a first mixer;
mixing the contents of the first mixer and reacting the first measured dose of the cementation reagents in the presence of the first measured dose of the at least one biological organism or enzyme to form a biocement which binds to the surfaces of the aggregate particles, thereby increasing the size of the particles to yield a biocement coated aggregate;
combining a second measured dose of at least one biological organism or enzyme and a second measured dose of cementation reagents with the biocement coated aggregate in either the first mixer or a second mixer;
compacting the mixture of the biocement coated aggregate, the second measured dose of at least one biological organism or enzyme, and the second measured dose of cementation reagents in a mold or form to reduce the volume of empty space between the biocement coated aggregate particles; and
reacting the second measured dose of cementation reagents in the presence of the second measured dose of at least one biological organism or enzyme to form biocement bridges between the biocement coated aggregate particles, thereby consolidating the biocement coated aggregate particles into a bioconcrete construction material.

2. The method of claim 1, wherein the first or second mixer is a thermally regulated to maintain a target temperature.

3. The method of any one of claims 1-2, wherein the total moisture content of the contents of the first or second mixer is less than about 25 wt %.

4. The method of any one of claims 1-3, wherein the total moisture content of the contents of the first or second mixer is controlled and to be less than about 15 wt %.

5. The method of any one of claims 1-4, wherein the steps of the method are carried out in a temperature-controlled and humidity-controlled environment.

6. The method of any one of claims 1-5, wherein the temperature, humidity, and/or pH are monitored and controlled during either cementation reaction.

7. The method of any one of claims 1-6, wherein either of the first or second measured doses of at least one biological organism or enzyme comprises urease or cells of a urease-producing microorganism.

8. The method of any one of claims 1-7, wherein either of the first or second measured doses of at least one biological organism or enzyme comprises an acid-producing enzyme or cells of an acid-producing microorganism.

9. The method of any one of claims 1-8, wherein either of the first or second measured doses of at least one biological organism or enzyme comprises carbonic anhydrase or cells of a carbonic anhydrase-producing microorganism.

10. The method of claim 7, wherein the urease-producing microorganism is Sporosarcina pasteurii.

11. The method of claim 7, wherein the urease-producing microorganism is selected from the group of: Sporosarcina pasteurii, Sporosarcina ureae, Proteus vulgaris, Bacillus sphaericus, Myxococcus xanthus, Proteus mirabilis, Bacillus megaterium, Helicobacter pylori, and combinations of two or more thereof.

12. The method of any one of claims 7-11, wherein the cells comprise spores.

13. The method of any one of claims 1-10, wherein the biocement comprises bacterially precipitated calcium carbonate.

14. The method of any one of claims 1-13, wherein either of the first or second measured dose of cementation reagents comprises nutrients which promote the growth or enzymatic activities of microorganisms.

15. The method of claim 14, wherein the nutrients comprise one or more of salts, amino acids, proteins, peptides, carbohydrates, saccharides, polysaccharides, fatty acids, oil, vitamins and minerals.

16. The method of any one of claims 1-15, wherein either of the first or second measured dose of cementation reagents comprises a calcium source.

17. The method of any one of claims 1-16, wherein either of the first or second measured dose of cementation reagents comprises a urea source.

18. The method of any one of claims 1-17, wherein either of the first or second measured dose of cementation reagents comprises calcium carbonate.

19. The method of any one of claims 1-18, wherein either of the first or second measured dose of cementation reagents comprises calcium chloride.

20. The method of any one of claims 1-19, wherein either of the first or second measured dose of cementation reagents comprises cells of a urea-producing microorganism.

21. The method of claim 20, wherein the urea-producing microorganism is selected from the group of: Pseudomonas, Delaya avenusta, Thiosphaera pantotropha, Pseudomonas stutzen, Fragilaria crotonensis, Pseudoalteromonas spp., Pseudoalteromonas haloplanktis, Halomonas venusta, Pseudomonas balearica, Pseudomonas stutzeri, Bacillus megaterium, Exiguobacterium aurantiacum, Pseudoalteromonas aliena, Pseudoalteromonas luteoviolacea, E. coli, and variants, serotypes, mutations, recombinant forms, and combinations thereof.

22. The method of claim 8, wherein the acid-producing microorganism is selected from the group consisting of: Variovorax, Klebsiella, Pseudomonas, Bacillus, Exiguobacterium, Microbacterium, Curtobacterium, Rathayibacter, CellFimi2, Streptomyces, Raoultella, B. pumilus, B. safanensis, B. simplex, B. licheniformis, and combinations thereof.

23. The method of claim 8 or 22, wherein the acid produced by the acid-producing enzyme of the cells of the acid-producing microorganism is a carboxylic acid.

24. The method of claim 8 or 22-23, wherein the acid produced by the acid-producing enzyme of the cells of the acid-producing microorganism is acetic acid, formic acid, propionic acid, butyric acid, gluconic acid, succinic acid, lactic acid, or citric acid.

25. The method of any one of claims 1-24, wherein compacting the mixture reduces the average linear distance between adjacent aggregate particles by at least about 25%.

26. The method of any one of claims 1-25, wherein compacting the mixture produces an absolute packing efficiency of the aggregate particles of at least about 50%.

27. The method of any one of claims 1-26, wherein compacting the mixture is performed by placing the mixture in a vibratory press and applying pressure and vibration to reduce the volume of empty space between the biocement coated aggregate particles.

28. The method of claim 27, wherein the vibratory motor of the press is operated at a rotational speed from about 100 RPM to about 7200 RPM.

29. The method of any one of claims 27-28, wherein the vibratory motor of the press is operated at a duty cycle from 0.01% to about 100%.

30. The method of any one of claims 27-29, wherein the vibration and pressure are applied simultaneously.

31. The method of any one of claims 27-29, wherein the vibration and pressure are applied alternatively.

32. The method of any one of claims 1-31, wherein the finished bioconcrete construction material comprises at least about 2% biocement by weight.

33. The method 1-32, wherein the finished bioconcrete construction material comprises at most about 20% biocement by weight.

34. The method of any one of claims 1-33, wherein the aggregate particles comprise natural, non-natural, recycled or manufactured sand, ore, crushed rock, stone, minerals, crushed or fractured glass, wood, ash, foam, basalt, fibers, mine tailings, paper, waste materials, waste from a manufacturing process, plastics, polymers, roughened materials, and/or combinations thereof.

35. The method of any one of claims 1-34, wherein the bioconcrete construction material comprises bricks, thin bricks, pavers, panels, tile, veneer, cinder, breeze, clinker or aerated blocks, counter-tops, table-tops, design structures, blocks, a solid masonry structure, piers, foundations, beams, walls, or slabs.

36. A mixing device for biocement processing, the mixing device comprising:
a mixing tank;
a plurality of mixing tines;
a mixing motor, operably coupled to the mixing tines;
a motor controller, operably coupled to the mixing motor;
a reagent delivery pump, operably coupled by a reagent line to infuse a quantity of a reagent from a reservoir into the mixing tank;
a pump controller, operably coupled to the reagent delivery pump;
a heater operably coupled to the mixing tank;
a temperature sensor configured to measure the temperature inside the mixing tank;
a temperature controller, operably coupled to the temperature sensor and the heater; and
a main controller, operably coupled to the motor controller, pump controller, and temperature controller;
wherein, the main controller is configured to facilitate production of biocement coated aggregate particles using the mixing device.

37. The mixing device of claim 33, wherein the motor controller is configurable to set the motor speed and the motor duty cycle.

38. The mixing device of any one of claims 33-37, wherein the motor controller is configurable to set a total mixing time.

39. The mixing device of any one of claims 33-38, wherein the temperature controller maintains a set temperature using a proportional-integral-derivative control algorithm based on a temperature value measured by the temperature sensor.

40. The mixing device of any one of claims 33-39, wherein the reagent delivery pump further comprises a second temperature sensor, a second heater, and is operably coupled to the temperature controller.

41. The mixing device of any one of claims 33-40, wherein the mixing motor can be configured to operate at speeds from about 1 to about 120 RPM.

42. The mixing device of any one of claims 33-41, wherein the duty cycle is configurable from about 0.01% to about 100%.

43. The mixing device of any one of claims 33-42, wherein the duty cycle is configurable from about 0.05% to about 0.1%.

44. The mixing device of any one of claims 33-43, wherein the temperature controller is configurable to maintain a temperature of about 20° C. to about 40° C.

45. The mixing device of any one of claims 33-44, wherein the mixing tank has a mixing capacity of about 0.1 yd³ to about 30 yd³.

46. The mixing device of any one of claims 33-45, wherein the mixing tank has a mixing capacity of at least about 4 yd³.

47. The mixing device of any one of claims 33-46, wherein the reagent delivery pump is configurable to deliver a fixed volume, a fixed mass, a fixed flow rate, or a custom flow pattern of reagent into the mixing tank.

48. The mixing device of any one of claims 33-47, further comprising a moisture sensor operably coupled to the main controller and configured to measure the moisture level inside the mixing tank.

49. The mixing device of claim 48, wherein the moisture level inside the tank is used to adjust the quantity of reagent delivered into the tank or is used to adjust the temperature controller.

50. The mixing device of any one of claims 33-49, wherein the heater is a heat-exchanger.

51. The mixing device of any one of claims 33-49, wherein the heater is a resistive heater.

52. The mixing device of any one of claims 33-49, wherein the heater is a forced-air heater.

53. The mixing device of any one of claims 33-52, wherein the device maintains a homogeneous temperature inside the mixing tank, reagent reservoir, and reagent line at a set point of the temperature controller, and is substantially free of hot spots.

54. The mixing device of any one of claims 33-53, wherein the reagent pump is a peristaltic pump, a syringe pump, a rotary vane pump, a venturi pump, or a diaphragm pump.

55. The mixing device of any one of claims 33-54, wherein the mixing tines are attached to the a mixing paddle or mixing wheel, which is mounted inside of the mixing tank and operably coupled to an output shaft of the mixing motor.

56. The mixing device of any one of claims 33-54, wherein the mixing tines are attached to the walls of the mixing tank and the mixing tank comprises a rotatable drum which is operably coupled to an output shaft of the mixing motor.

57. The mixing device of any one of claims 33-56, wherein the reservoir is configured to deliver a reagent comprising a biological organism or enzyme, calcium, and urea.

58. The mixing device of any one of claims 33-57, wherein the mixing tank further comprises a pH sensor or ion selective electrode for reaction monitoring, operably coupled to the main controller.

59. A construction material comprising aggregate particles, calcium carbonate, and a supplemental material, wherein the mass ratio of the supplemental material to the calcium carbonate is no more than about 1:1, and wherein the construction material has a compressive strength which is increased at least about 10% relative to an otherwise identical construction material wherein the supplemental material is not present.

60. A construction material comprising aggregate particles, calcium carbonate, and about 0.1 weight % to about 40 weight % of a supplemental material, and wherein the construction material has a compressive strength which is increased at least about 10% relative to an otherwise identical construction material wherein the supplemental material is not present.

61. A construction material comprising aggregate particles, calcium carbonate, and about 0.1 weight % to about 40 weight % of a supplemental material, and wherein the construction material has a compressive strength which is at least about 900 psi.

62. A construction material comprising aggregate particles, calcium carbonate, and a supplemental material, wherein the mass ratio of the supplemental material to the calcium carbonate is no more than about 1:1, and wherein the construction material has a compressive strength which is at least about 900 psi.

63. The construction material of any of claims 59-62, wherein the supplemental material is a metal sulfate.

64. The construction material of any of claims 59-63, wherein the supplemental material is a metal silicate.

65. The construction material of any of claims 59-64, wherein the supplemental material is a metal hydroxide.

66. The construction material of any of claims 59-65, wherein the supplemental material is calcium sulfate.

67. The construction material of any of claims 59-66, wherein the supplemental material is calcium hydroxide.

68. The construction material of any of claims 59-67, wherein the supplemental material is calcium oxide.

69. The construction material of any of claims 59-68, wherein the supplemental material is a bentonite clay.

70. The construction material of any of claims 59-69, wherein the supplemental material is a mixture comprising one or more components selected from the group of metal silicates, metal carbonates, metal sulfates, metal oxides, and metal hydroxides.

71. The construction material of any of claims 59-70, wherein the supplemental material is a mixture comprising calcium oxide, silica, alumina, iron oxide, magnesium oxide, and sulfites (e.g. Ordinary Portland cement).

72. The construction material of any of claims 59-71, further comprising fossilized cells of a microorganism.

73. The construction material of any of claims 59-72, wherein the mass ratio of the supplemental material to the calcium carbonate is no more than about 0.2:1.

74. The construction material of any of claims 59-73, wherein the calcium carbonate is from about 0.01 weight % to about 20 weight % of the construction material.

75. The construction material of any of claims 59-74, wherein a carbon footprint of the construction material is at least about 60% less than that of a functionally equivalent construction material made from ACI 318 structural concrete.

76. The construction material of any of claims 59-75, wherein a carbon footprint of the construction material is at least about 80% less than that of a functionally equivalent construction material made from ACI 318 structural concrete.

77. The construction material of any of claims 59-76, wherein a carbon footprint of the construction material is at least about 90% less than that of a functionally equivalent construction material made from ACI 318 structural concrete.

78. The construction material of any of claims 59-77, wherein a carbon footprint of the construction material is at least about 95% less than that of a functionally equivalent construction material made from ACI 318 structural concrete.

79. A method of making the construction material of any of claims 59-78, the method comprising:
combining aggregate particles with biocementation reagents and a supplemental material to yield a biocement mixture; and
reacting the cementation reagents in the presence of the supplemental material to produce a biocement, thereby yielding the construction material.

80. The method of claim 79, wherein the steps of the method are carried out in a temperature-controlled and humidity-controlled environment.

81. The method of any one of claims 79-80, wherein a temperature, humidity, and/or pH are monitored and controlled during the reacting.

82. The method of any one of claims 79-81, wherein the biocementation reagents comprise at least one biological organism or enzyme further comprising urease or cells of a urease-producing microorganism.

83. The method of any one of claims 79-82, wherein the biocementation reagents comprise an acid-producing enzyme or cells of an acid-producing microorganism.

84. The method of any one of claims 79-83, wherein the biocementation reagents comprise carbonic anhydrase or cells of a carbonic anhydrase-producing microorganism.

85. The method of claim 82, wherein the urease-producing microorganism is Sporosarcina pasteurii.

86. The method of claim 82, wherein the urease-producing microorganism is selected from the group of: Sporosarcina pasteurii, Sporosarcina ureae, Proteus vulgaris, Bacillus sphaericus, Myxococcus xanthus, Proteus mirabilis, Bacillus megaterium, Helicobacter pylori, and combinations of two or more thereof.

87. The method of any one of claims 82-86, wherein the cells comprise spores.

88. The method of any one of claims 79-87, wherein the biocementation reagents comprise nutrients which promote the growth or enzymatic activities of microorganisms.

89. The method of claim 88, wherein the nutrients comprise one or more of salts, amino acids, proteins, peptides, carbohydrates, saccharides, polysaccharides, fatty acids, oil, vitamins and minerals.

90. The method of any one of claims 79-89, the biocementation reagents comprise a calcium source.

91. The method of any one of claims 79-90, wherein the biocementation reagents comprise a urea source.

92. The method of any one of claims 79-91, wherein the biocementation reagents comprise calcium carbonate.

93. The method of any one of claims 79-92, wherein the biocementation reagents comprise calcium chloride.

94. The method of any one of claims 79-93, wherein the biocementation reagents comprise cells of a urea-producing microorganism.

95. The method of claim 94, wherein the urea-producing microorganism is selected from the group of: Pseudomonas, Delaya avenusta, Thiosphaera pantotropha, Pseudomonas stutzen, Fragilaria crotonensis, Pseudoalteromonas spp., Pseudoalteromonas haloplanktis, Halomonas venusta, Pseudomonas balearica, Pseudomonas stutzeri, Bacillus megaterium, Exiguobacterium aurantiacum, Pseudoalteromonas aliena, Pseudoalteromonas luteoviolacea, E. coli, and variants, serotypes, mutations, recombinant forms, and combinations thereof.

96. The method of claim 83, wherein the acid-producing microorganism is selected from the group consisting of: Variovorax, Klebsiella, Pseudomonas, Bacillus, Exiguobacterium, Microbacterium, Curtobacterium, Rathayibacter, CellFimi2, Streptomyces, Raoultella, B. pumilus, B. safanensis, B. simplex, B. licheniformis, and combinations thereof.

97. The method of claim 83 or 96, wherein the acid produced by the acid-producing enzyme of the cells of the acid-producing microorganism is a carboxylic acid.

98. The method of claim 83 or 96-97, wherein the acid produced by the acid-producing enzyme of the cells of the acid-producing microorganism is acetic acid, formic acid, propionic acid, butyric acid, gluconic acid, succinic acid, lactic acid, or citric acid.

99. The method of any one of claims 79-98, further comprising compacting the mixture to reduce the average linear distance between adjacent aggregate particles by at least about 25%.

100. The method of any one of claims 79-99, further comprising compacting the mixture to produce an absolute packing efficiency of the aggregate particles of at least about 50%.

101. The method of any one of claims 79-100, further comprising compacting the mixture by placing the mixture in a vibratory press and applying pressure and vibration to reduce the volume of empty space between a plurality of biocement coated aggregate particles.

102. The method of claim 101, wherein the vibratory motor of the press is operated at a rotational speed from about 100 RPM to about 7200 RPM.

103. The method of any one of claims 101-102, wherein the vibratory motor of the press is operated at a duty cycle from 0.01% to about 100%.

104. The method of any one of claims 101-103, wherein the vibration and pressure are applied simultaneously.

105. The method of any one of claims 101-103, wherein the vibration and pressure are applied alternatively.

106. The method of any one of claims 79-105, wherein the finished construction material comprises at least about 2% biocement by weight.

107. The method of any of claim 1-35 or 79-106, wherein the finished construction material comprises at most about 20% biocement by weight.

108. The construction material or the method of any of the preceding claims, wherein the aggregate particles comprise natural, non-natural, recycled or manufactured sand, ore, crushed rock, stone, minerals, crushed or fractured glass, wood, ash, foam, basalt, fibers, mine tailings, paper, waste materials, waste from a manufacturing process, plastics, polymers, roughened materials, and/or combinations thereof.

109. The construction material or the method of any of the preceding claims, wherein the construction material comprises bricks, thin bricks, pavers, panels, tile, veneer, cinder, breeze, clinker or aerated blocks, counter-tops, table-tops, design structures, blocks, a solid masonry structure, piers, foundations, beams, walls, or slabs.

110. The construction material or the method of any of the preceding claims, wherein the construction material has a compressive strength which is increased at least about 25% relative to an otherwise identical construction material wherein the supplemental material is not present.

111. The construction material or the method of any of the preceding claims, wherein the construction material comprises about 0.5% weight % to about 5 weight % of a supplemental material, and wherein the construction material has a compressive strength which is increased at least about 25% relative to an otherwise identical construction material wherein the supplemental material is not present.

112. The construction material or the method of any of the preceding claims, wherein the construction material comprises about 0.5% weight % to about 5 weight % of a supplemental material, and wherein the construction material has a compressive strength which is at least about 900 psi.
